(19) United States
(12) Patent Application Publication
(10) Pub. No.: US 2024/0342244 A1
(43) Pub. Date: Oct. 17, 2024

(54) Isthmin Protein Therapeutics for the Treatment of Non-Alcoholic Fatty Liver Disease

(71) Applicant: The Board of Trustees of the Leland Stanford Junior University, Stanford, CA (US)

(72) Inventors: Katrin Jennifer Svensson, Palo Alto, CA (US); Laetitia Voilquin, Redwood City, CA (US)

(21) Appl. No.: 18/292,488

(22) PCT Filed: Jul. 27, 2022
(86) PCT No.: PCT/US2022/074207

§ 371 Date: Jan. 26, 2024

Related U.S. Application Data
(60) Provisional application No. 63/226,600, filed on Jul. 28, 2021.

(51) Int. Cl. A61K 38/19; A61K 31/155; A61K 45/06; A61P 1/16; A61P 3/10

(52) U.S. Cl. CPC A61K 38/19; A61K 31/155; A61K 45/06; A61P 1/16; A61P 3/10

(57) ABSTRACT

Compositions and methods are provided for the treatment of one or both of type 2 diabetes and fatty liver disease, e.g. non-alcoholic fatty liver disease (NAFLD) and nonalcoholic steatohepatitis (NASH). The adipokine Isthmin-1 (ISM1) protein increases adipose glucose uptake while suppressing hepatic lipid synthesis.

### CROSS-REFERENCE

This application claims the benefit of U.S. Provisional Patent Application No. 63/226,600 filed Jul. 28, 2021, which application is incorporated herein by reference in its entirety.

### GOVERNMENT RIGHTS

This invention was made with Government support under contract DK125260 awarded by the National Institutes of Health and National Institute of Diabetes and Digestive and Kidney Diseases. The Government has certain rights in this invention.

### INCORPORATION BY REFERENCE OF SEQUENCE LISTING PROVIDED AS A TEXT FILE

A Sequence Listing is provided herewith as a Sequence Listing XML, “S21-259_STAN-1874WO_Sequence listing” created on Jul. 27, 2022 and having a size of 3 KB. The contents of the Sequence Listing XML are incorporated by reference herein in their entirety.

### BACKGROUND

The growing epidemic of metabolic disorders has increased the need for a greater mechanistic understanding of the molecular basis for glucose and lipid regulation in normal physiology and pathophysiology. Glucose homeostasis balances glucose uptake, mainly by skeletal muscle, heart and adipose tissue, and glucose production, predominantly by the liver. Activation of brown or beige fat in humans has been shown to increase both basal and insulin-stimulated whole-body glucose disposal, demonstrating a physiologically significant role for these tissues in glucose regulation. By using ¹⁸F-fluoro-2-deoxy-d-glucose positron emission tomography (18F-FDG-PET), several groups have shown that glucose uptake into thermogenic adipose tissues in humans can be induced by cold exposure or by pharmacological activation of the β-adrenergic receptors. These findings are in agreement with studies in rodents showing that loss of the futile creatine cycle from adipose tissues causes obesity and worsens glucose tolerance. Furthermore, brown fat transplanted into the visceral cavity of mice can improve glucose tolerance and improve insulin sensitivity.

There is mounting evidence that thermogenic adipose tissue can mediate some of the beneficial effects through secreted factors, but the molecules and pathways remain incompletely understood. Several studies have identified autocrine or paracrine mediators of glucose metabolism, including fibroblast growth factor 21 (FGF21), interleukin-6 (IL-6), slit2-C and neuregulin 4.

Two pathways have been proposed to increase glucose uptake in adipose tissue: insulin-dependent glucose uptake during anabolic processes and insulin-independent glucose uptake by molecules such as fibroblast growth factor 21 (FGF21), or norepinephrine during thermogenesis. Importantly, insulin promotes lipid synthesis, a process that contributes to the development of non-alcoholic fatty liver disease. As a consequence, hyperinsulinemia further exacerbates the metabolic triad of hyperglycemia, hypertriglyceridemia and insulin resistance. Current insulin therapies and insulin-sensitizing agents are often associated with undesirable effects, such as increases in hepatic lipid synthesis. Identifying pathways to simultaneously increase peripheral glucose uptake while suppressing hepatic lipid accumulation would be beneficial for an overall improvement of metabolic syndrome. Interestingly, mice lacking beige adipose tissue have worsened hepatic steatosis, suggesting the existence of fat-derived paracrine factors that can regulate lipid accumulation.

Non-alcoholic fatty liver disease (NAFLD) and non-alcoholic steatohepatitis (NASH) are increasing global health problems with substantial unmet medical needs, particularly considering the lack of FDA-approved treatments. The present disclosure addresses this problem.

### SUMMARY

ISM1 is a biologically bioactive ligand that is shown herein to dissociate glucose uptake from lipid synthesis, providing new therapeutic avenues for glucose and lipid-associated disorders. Therapeutic administration of ISM1 protein to an individual in need thereof improves diabetes, e.g. decreasing insulin resistance, and ameliorating hepatic steatosis.

Compositions and methods are provided for the treatment of one or both of type 2 diabetes, and fatty liver disease, e.g. non-alcoholic fatty liver disease (NAFLD) and nonalcoholic steatohepatitis (NASH) by administration of an effective dose of ISM1 protein. In some embodiments, the methods disclosed herein treat hyperglycemia and hyperlipidemia simultaneously. It is shown herein that the adipokine Isthmin-1 (ISM1) protein increases adipose glucose uptake while suppressing hepatic lipid synthesis. ISM1 counteracts lipid accumulation in the liver by switching hepatocytes from a lipogenic to a protein synthesis state.

In some embodiments, a method of preventing or reducing symptoms of fatty liver disease, e.g. NAFLD, NASH, etc., are provided, the method comprising administering an effective dose of an ISM1 agent for a period of time sufficient to prevent or reduce symptoms of fatty liver disease. In some embodiments the ISM1 agent is ISM1 protein, e.g. human ISM1 protein or a variant thereof. In some embodiments the effective dose is at least about 0.1 mg/kg, at least about 0.5 mg/kg, at least about 1 mg/kg, at least about 5 mg/kg, at least about 10 mg/kg, at least about 20 mg/kg, at least about 50 mg/kg, at least about 100 mg/kg, in some embodiments the effective dose is from about 1 to 50 mg/kg. Dosing may be daily, every 2 days, every 3 or more days, e.g. weekly, semi-weekly, bi-weekly, monthly, etc. Dosing may be parenteral, including sustained release formulations.

In some embodiments the methods of treatment provide for a reduction in one or more disease indicia of fatty liver disease. Indicia of effective disease treatment may include, without limitation, reduction of liver weight or mass; reduction of blood glucose, e.g. basal or stable overnight glucose concentration; reduced hepatic Fatty Acid Synthase (FAS) protein levels; reduction of hepatic steatosis, e.g. by histologic signs, ultrasound B mode examination, vibration-controlled transient elastography (VCTE), controlled attenuation parameter, and the like.

In some embodiments an ISM1 agent is administered in combination with a second agent useful in the treatment of glucose and lipid-associated disorders, e.g. metformin, sulfonylureas, e.g. glyburide, glipizide, glimepiride; Glinides, e.g. repaglinide and nateglinide; Thiazolidinediones, e.g. rosiglitazone, pioglitazone; DPP-4 inhibitors, e.g. sitagliptin, saxagliptin, linagliptin; GLP-1 receptor agonists, e.g. exenatide, liraglutide, semaglutide; SGLT2 inhibitors, e.g. canagliflozin, dapagliflozin, empagliflozin, etc. The mechanism of ISM1 action is distinct from any of the other currently utilized drug pathways, providing for complementarity and possible synergy in activity.

In some embodiments a formulation is provided, comprising an effective dose of an ISM1 agent, e.g. human ISM1 protein or variant thereof, and a pharmaceutically acceptable excipient. In some embodiments an ISM1 protein is a recombinant protein. In some embodiments the formulation comprises an effective unit dose of an ISM1 protein.

Without being limited by the theory, the data indicate that ISM1 activates a PI3K/AKT signaling pathway independently of the insulin receptors. While the glucoregulatory function is shared with insulin, ISM1 counteracts lipid accumulation in the liver by switching hepatocytes from a lipogenic to a protein synthesis state. ISM1 acts directly on hepatocytes in the presence of insulin to upregulate anabolic protein signaling pathways and protein synthesis, while suppressing srebp1c target genes and lipid synthesis. ISM1 does not display any activity or displays only weak activity on other pathways such as protein kinase A (PKA), PDK1 or GSK3β.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. ISM1 is a secreted protein that induces glucose uptake in human and mouse adipocytes. (A) LC-MS analysis detection of mouse ISM1 peptides in red from adipocyte conditioned media (n=2). (B) LC-MS analysis using TMT-labeling demonstrating relative ISM1 protein secretion in conditioned media from wt (n=2) or ap2-prdml6tg (n=2) adipocytes. (C) qRT-PCR of ism1, adiponectin, pparg and pdgfr-a in isolated mature brown fat adipocytes and in the stromal vascular fraction (n=3). (D) Western blot of Ism1 and b-actin in isolated mature brown fat adipocytes and in the stromal vascular fraction. Recombinant ISM1 protein was used as standard. (E) Ism1 gene expression levels in brown fat adipocytes during differentiation (n=2-3). (F) Silverstain and ISM1 immunoblot of native and deglycosylated recombinant mouse ISM1. (G) Size exclusion chromatography of recombinant ISM1 protein under native conditions. (H) LC-MS analysis of recombinant ISM1 protein showing high purity by spectral counts and number of unique peptides. (I) 2-deoxy-H³-glucose uptake in human SGBS adipocytes treated with insulin or ISM1 protein for 30 minutes (n=6). (J) 2-deoxy-H³-glucose uptake in mouse primary adipocytes treated with control or ISM1 across four biologically independent experiments (n=15). (K) 2-deoxy-H³-glucose uptake in mouse primary adipocytes treated with insulin, ISM1, or a combination of ISM1 and insulin for 1 h (n=3). (L) 2-deoxy-H³-glucose uptake in human primary skeletal muscle cells treated with ISM1 or insulin for 1 h (n=3). (M) Membrane localization of GLUT4 in primary adipocytes after treatment with 100 nM insulin or 100 nM ISM1 protein for 24 hours. (N) Protein levels of GLUT4 in isolated plasma membranes in primary mouse adipocytes treated with 100 nM ISM1 protein or 100 nM insulin for 4 h compared with the cytosolic fractions. PDGFR-α is used as loading control. (O) Ism1 and adiponectin gene expression levels in lacZ-shRNA or Ism1-shRNA adipocytes (n=4). (P) 2-deoxy-H³-glucose uptake in lacZ-shRNA or Ism1-shRNA adipocytes (n=4). (Q) Western blot of pAKT^{S473}, total AKT and β-actin in lacZ-shRNA or ISM1-shRNA adipocytes. (R) Quantification of protein expression pAKT^{S473}/total AKT quantified from two independent experiments in lacZ-shRNA or Ism1-shRNA adipocytes. (S) 2-deoxy-H³-glucose uptake in ISM1-shRNA adipocytes treated with indicated concentrations of insulin for 1 h compared with lacZ-shRNA (n=3). (T) Western blot of pAKT^{S473}, total AKT, and β-actin in lacZ-shRNA or ISM1-shRNA adipocytes treated with indicated concentrations of insulin for 5 min. (U) Quantification of protein expression pAKT^{S473}/total AKT quantified from two independent experiments. Data are presented as mean±S.E.M of biologically independent samples. * p<0.05, ** p<0.01, *** p<0.001 by two-tailed Student's t-test or Sidak's multiple comparisons test for multiple comparisons.

FIG. 2. Ablation of ISM1 results in glucose intolerance and impaired adipocyte glucose uptake. (A) Ism1 gene expression in different tissues from WT and ISM1-KO mice (n=5-8). (B) ISM1 serum levels in male WT and ISM1-KO mice using an ISM1 ELISA (n=5-8). (C) Body temperature in male WT and ISM1-KO mice (n=5-8). (D) Food intake in male WT and ISM1-KO mice (n=5-8). (E) Insulin levels in male WT and ISM1-KO mice (n=5-8). (F) Body weights in male WT and male WT and ISM1-KO mice (n=5-6). (G) Insulin tolerance test in male WT and ISM1-KO mice (n=10-14). (H) Glucose tolerance test in male WT and ISM1-KO mice (n=10-14). (I) In vivo 2-deoxy-H³-glucose uptake in brown adipose tissue (BAT), white inguinal adipose tissue (iWAT), quadriceps skeletal muscle (Quad), and liver from three cohorts of mice on chow, HFD or NASH diet (n=5-8). (J) In vivo 2-deoxy-H³-glucose uptake in brown adipose tissue (BAT) in male WT and ISM1-KO mice under basal and insulin-stimulated conditions (n=5). (K) In vivo 2-deoxy-H³-glucose uptake in brown adipose tissue (BAT), white inguinal adipose tissue (iWAT), quadriceps skeletal muscle (Quad), and liver in male ISM1-KO mice treated with 5 mg/kg ISM1 protein for two days (n=6-7). (L) Ism1 and adiponectin gene expression in differentiated adipocytes isolated from WT and Ism1-KO (n=4). (M) Oil red O staining of differentiated adipocytes isolated from WT and ISM1-KO mice (n=3). (N) Western blot of pAKT^{S473}, total AKT, and β-actin in mouse adipocytes isolated from WT and ISM1-KO mice. (O) Quantification of protein expression pAKT^{S473}/total AKT quantified from two independent experiments. (P) Western blot of pAKT^{S473}, total AKT, and β-actin in mouse adipocytes isolated from WT and ISM1-KO mice treated with indicated concentrations of insulin. (Q) 2-deoxy-H³-glucose uptake in differentiated adipocytes isolated from WT and ISM1-KO mice treated with indicated concentrations of insulin (n=4). Data are presented as mean±S.E.M of biologically independent samples. * p<0.05, ** p<0.01, *** p<0.001 by one- or two-tailed Student's t-test.

FIG. 3. ISM1 activates the PI3K/AKT pathway. (A) Phosphokinase array quantification of 3T3-F442A cells treated with vehicle (control) or 100 nM ISM1. (B) Western blot of pAKT^{S473} and AKT in 3T3-F442A cells treated with vehicle (ctl) ISM1-flag, ISM1-his, Thrombospondin-his (TSP-his) or insulin at 100 nM. (C) Western blot of pAKT^{S473}, AKT, and β-actin in mouse BAT adipocytes treated with indicated concentrations of ISM1 or insulin. (D) Western blot of pAKT^{S473}, AKT, and β-actin in mouse adipocytes treated with indicated concentrations of ISM1 or insulin. (E) Western blot of pAKT^{S473} and AKT in mouse adipocytes treated with 100 nM recombinant ISM1 or 10 nM insulin at different time points. (F) Western blot of pAKT^{S473}, AKT, and β-actin in human SGBS adipocytes treated with indicated concentrations of ISM1 or insulin. (G) Western blot of pAKT^{S473} and AKT in 3T3-F442A cells treated with ISM1 or insulin with indicated concentrations. (H) AlphaLISA SureFire Ultra AKT 1/2/3 (pS473) measurements in 3T3-F442A cells treated with recombinant ISM1 or insulin. (I) Western blot of 3T3-F442A cells treated with 100 nM ISM1, 100 nM insulin, or 20 ng/ml PDGF-ββ showing the intracellular signaling pathways over time. Data are presented as mean±S.E.M of biologically independent samples. * p<0.05, ** p<0.01, *** p<0.001 by two-tailed Student's t-test. For western blots, all protein treatment are 5 mins unless indicated otherwise.

FIG. 4. ISM1 signaling is independent of the Insulin and IGF receptors. (A) pAKT^{S473} signaling induced by ISM1 (100 nM) or insulin (100 nM) in 3T3-F442A cells pre-treated with PI3K inhibitors Wortmannin, LY294002, or PIK-75, mTORC1 inhibitor Rapamycin, mTORC1/2 dual inhibitor Torini, or the PI3K/mTOR inhibitor Omipalisib. (B) 2-deoxy-H³-glucose uptake in mouse primary adipocytes treated with recombinant mouse ISM1 protein in the absence or presence of 1 uM Wortmannin (n=3). (C) Signaling induced by ISM1 (100 nM) or insulin (100 nM) in 3T3-F442A cells pre-treated for 30 min with the selective mTOR inhibitor INK-128. (D) Signaling induced by ISM1 (100 nM) or insulin (100 nM) in 3T3-F442A cells pre-treated for 30 min with S6K inhibitor DG2. (E) Western blot of pAKT^{S473}, AKT, and β-actin in 3T3-F442A cells treated with insulin in the presence of 0 nM, 25 nM or 50 nM ISM1. (F) Western blot of phosphorylated insulin and IGF receptors in 3T3-F442A cells treated with 100 nM ISM1 or 100 nM insulin for 2 min. (G) Signaling induced by ISM1 (100 nM) or insulin (100 nM) in 3T3-F442A cells pre-treated for 30 min with the IR/IGFR inhibitor OSI-906. (H) 2-deoxy-H³-glucose uptake in human SGBS adipocytes treated with ISM1 (100 nM) in the absence or presence of 50 nM OSI-906 (n=3). (I) Schematic illustration of the signaling pathway activated by ISM1. For western blots, all protein treatment are 5 mins unless indicated otherwise. Data are presented as mean±S.E.M of biologically independent samples. * p<0.05, ** p<0.01, *** p<0.001 by two-tailed Student's t-test.

FIG. 5. ISM1-AAV overexpression prevents insulin resistance and hepatic steatosis in DIO mice. (A) Overview of prophylactic ISM1 overexpression in diet-induced obese (DIO) mice fed a HFD at the start of the experiment. (B) Western blot of plasma from AAV8-GFP and AAV8-ISM1-flag using an anti-FLAG antibody detecting the C-terminal flag tag of ISM1 (n=3). (C) Body weights in AAV8-GFP and AAV8-ISM1 mice measured during 10 weeks of HFD (n=10). (D-F) Glucose tolerance test (D), Insulin sensitivity test (E), plasma insulin levels (F) in mice expressing AAV8-GFP and AAV8-ISM1 after 10 weeks of HFD (n=10). (G) Plasma glucose levels during hyperinsulinemic-euglycemic clamp in AAV8-GFP and AAV8-ISM1 mice at 3 weeks of HFD (n=5-8). (H) Glucose infusion rate (GIR) during hyperinsulinemic-euglycemic clamp in AAV8-GFP and AAV8-ISM1 mice at 3 weeks of HFD (n=5-8). (I) Basal endogenous glucose production in AAV8-GFP and AAV8-ISM1 mice at 3 weeks HFD (n=5-8). (J) Endogenous glucose production under clamped conditions in AAV8-GFP and AAV8-ISM1 mice at 3 weeks of HFD (n=5-8). (K) H&E and Oil red O staining in livers from AAV8-GFP and AAV8-ISM1 mice. (L) Gene expression levels of hepatic lipogenesis genes in AAV8-GFP and AAV8-ISM1 mice (n=10). (M) Liver triglycerides quantification in AAV-GFP and AAV8-ISM1 mice after 10 weeks on HFD (n=10). Data are presented as mean±S.E.M of biologically independent samples. * p<0.05, ** p<0.01, *** p<0.001 by two-tailed Student's t-test or two-way Anova for repeated measurements.

FIG. 6. ISM1 suppresses de novo lipogenesis and promotes protein synthesis in hepatocytes. (A) Gene expression levels of Ism1 and srebp1c in primary hepatocytes overexpressing lacZ or ISM1 (n=3). (B) H³-acetate incorporation into lipids (de novo lipogenesis) in AML12 hepatocytes treated with ISM1 for 24 h in the presence or absence of insulin (n=3). (C) Western blot of SREBP1c, FAS, ACC and β-actin in AML12 hepatocytes treated with ISM1 for 24 h in the presence or absence of insulin. (D-G) Gene expression of lipogenic genes in AML12 hepatocytes after 6 h treatment with ISM1 in the presence or absence of insulin. (H) Western blot of S6^{S235/236}, AKT^{S473}, AKT and b-actin in AML12 hepatocytes treated with ISM1 for 24 h in the presence or absence of insulin. (I) Quantification of S6^{S235/236} relative b-actin in control, 50 nM ISM1 and 100 nM ISM1 from combined treatments in H. (J) H³-leucine incorporation into proteins (protein synthesis) in AML12 hepatocytes treated with ISM1 for 24 h in the presence or absence of 50 nM (n=3). (K) Fold change of substrate incorporation as a measure of lipogenesis or protein synthesis in AML12 hepatocytes treated with 50 nM insulin alone or 100 nM ISM1 and 50 nM insulin combined for 24 h (n=3). Data are presented as mean±S.E.M of biologically independent samples. * p<0.05, ** p<0.01, *** p<0.001 by two-tailed Student's t-test or Tukey's multiple comparisons test.

FIG. 7. Therapeutic administration of recombinant ISM1 improves glucose tolerance and hepatic steatosis. (A) Pharmacokinetic levels of serum ISM1-his using an anti-his-ELISA after I.V. injection of 10 mg/kg ISM1 in C5BL/6J mice. (B) Western blot of pAKTS473, total AKT, β-actin or tubulin in metabolic tissues after a single I.V. injection of 10 mg/kg recombinant ISM1 or 1 U/kg insulin in 16 weeks DIO C5BL/6J male mice. (C) Overview of therapeutic administration of ISM1 protein by daily I.P. injections of 5 mg/kg ISM1, or oral administration of 100 mg/kg metformin, or a combination of both into 12 weeks DIO mice for 21 days. (D-H) Body weights (D), Food intake (E), fasting blood glucose (F), GTT (G), and ITT (H) after 21 days of daily administration of either vehicle, 5 mg/kg ISM1, 100 mg/kg metformin, or a combination of 5 mg/kg ISM1 and 100 mg/kg metformin. (I) Overview of therapeutic administration with vehicle, 5 mg/kg ISM1, 0.5 mg/kg ISM1, or 30 mg/kg GW4064 for 14 days in NAFLD-induced mice. (J-L) Body weights (J), liver weights (K), fed blood glucose (L) of chow (control) mice, or mice with NAFLD treated with either vehicle, 5 mg/kg ISM1, 0.5 mg/kg ISM1, or 30 mg/kg GW4064 for 14 days (n=5). (M) Macroscopic liver photographs from representative mice treated with vehicle or 5 mg/kg ISM1 for 14 days. (N) Liver Fatty acid synthase (FAS) protein levels in chow (control) mice, or mice with NAFLD treated with vehicle, 5 mg/kg ISM1, 0.5 mg/kg ISM1, or 30 mg/kg GW4064 for 14 days (n=5). (O) H&E and Oil red O (lipid) staining in livers from chow (control) mice, or mice with NAFLD treated with vehicle, 5 mg/kg ISM1, 0.5 mg/kg ISM1, or 30 mg/kg GW4064 for 14 days (n=5). Data are presented as mean±S.E.M of biologically independent samples. * p<0.05, ** p<0.01, *** p<0.001 by two-tailed Student's t-test and Two-Way Anova for repeated measurements (GTT and ITT).

FIG. 8. Ism1 is a secreted protein that induces glucose uptake in human and mouse adipocytes (A) Heatmap of relative expression levels of predicted secreted genes from adiponectin-TRAP mice (n=3). (B) Ism1 gene expression levels in iWAT and BAT (n=5). 1 technical replicate of 5 biological samples. (C-D) qRT-PCR of Ism1 in BAT (C) or iWAT (D) from wt or ap2-prdml6tg mice (n=9-10). 1 technical replicate of 9-10 biological samples. (E-F) Ism1 and Ucp1 gene expression levels in brown fat (E) and iWAT (F) from 6-week-old C57BL/6 male mice in 30° C. thermoneutrality or after 14 days of 4° C. cold exposure (n=5). 1 technical replicate of 5 biological samples. (G) Representative western blot (n=2 in total) of mouse recombinant Ism1 protein. (H) Endotoxin levels <0.1 EU/ml in the recombinant Ism1 protein (n=3). (I) Protein aggregation test of 0.15 mg/ml Ism1 or insulin using transmittance (n=3). (J) Ucp1 gene expression levels in mouse adipocytes after treatment with 50, 100, or 200 nM Ism1 for 24 h or with forskolin for 1 h (n=3 biological replicates). (K) Crt, Gatm, Gamt, Ckmt1, Serca2b, Pm20d1 gene expression levels in adipocytes after 24 h Ism1 (n=4 biological replicates). (L) Cellular respiration expressed as pmoles/min under basal conditions or treated with 100 nM recombinant Ism1, 100 nM norepinephrine (NE), FCCP or rotenone (n=3 biological replicates). (M) Lipolysis assay in mouse primary adipocytes after 4 h treatment with Ism1, insulin or Isoproterenol (n=3 biological replicates). (N) 2-deoxy-³H-glucose uptake in primary adipocytes treated with 100 nM Ism1 protein or 100 nM albumin (control) (n=4 biological replicates). (O) 2-deoxy-³H-glucose uptake in 3T3-L1 cells treated with Ism1 or insulin for 1 h (n=3 biological replicates). Data are presented as mean±S.E.M of biologically independent samples. *P<0.05, **P<0.01, ***P<0.001, ns=nonsignificant, by two-tailed Student's t-test (c, d, e, f, j, k, m, n, o).

FIG. 9. Ism1 expression is correlated with obesity in mice and humans (A-B) Leptin and Ism1 gene expression in iWAT (A) or BAT (B) tissues in C57BL/6J male lean or 16-week DIO mice (n=5 biological replicates). (C) Regression analysis of ISM1 RNA expression levels (CPM) in human iWAT tissues correlated with body mass index (BMI) (n=43 biological replicates). (D) RNA expression levels (CPM) of ISM1 in iWAT tissue from patients stratified based on BMI (n=43 biological replicates). (E-F) RNA expression levels (CPM) of ISM1 in patients with glucose levels higher or lower than 100 mg/dl (E), or plasma insulin levels higher or lower than 10 ulU/ml (F) (n=43 biological replicates). (G-J) Regression analysis of ISM1 RNA expression levels (CPM) in human iWAT tissues correlated with insulin levels (G), glucose levels (H), HOMA-IR (I), or free fatty acid (FFA) levels (J) (n=43 biological replicates). (K-L) Regression analysis of circulating plasma human ISM1 levels correlated with BMI (n=11 biological replicates) (K) or glucose levels (n=43 biological replicates) (L). Data are presented as mean±S.E.M of biologically independent samples. *P<0.05, **P<0.01, ***P<0.001 by two tailed Student's t-test (a, b), regression analysis (c, g, g, l, j) or by Mann-Whitney test (d, e, f).

FIG. 10. Ablation of Ism1 results in glucose intolerance and impaired adipocyte glucose uptake. (A) Strategy for generation of the Ism1 floxed allele and Ism1 knockout (KO) allele by Crispr-Cas9. (B) Sequencing of the genetic recombination of Exon 1 and 5. (C) PCR product of the genetic recombination of Exon 1 and 5. (D) Genotyping of wild-type (WT), Ism1 heterozygous (Het), or Ism1-KO in the Ella-Cre transgenic background. (E) In vivo 2-deoxy-H3-glucose uptake in white inguinal adipose tissue (iWAT), quadriceps skeletal muscle (Quad), and liver in male WT and Ism1-KO mice under basal and insulin-stimulated conditions (n=5 biological replicates). (F) Protein expression (FIG. 2P) quantified from two independent western blot experiments of pAKT^{S473} over total AKT in primary mouse adipocytes isolated from WT and Ism1-KO mice treated with insulin (n=2 in total). Data are presented as mean±S.E.M of biologically independent samples. *P<0.05, **P<0.01, ***P<0.001, ns=nonsignificant, by two-tailed Student's t-test (e, f).

FIG. 11. Ism1 activates a PI3K-AKT pathway. (A) Family tree analysis of mouse Ism1 by protein blast using Ism1 NCBI sequence gene ID: 319909. (B) Representative western blot (n=2 in total) of pAKT^{S473} induced by Ism1 (100 nM) or insulin (100 nM) in C2C12 cells at 5 min. (C) Western blot (n=1 biological replicate) of pAKT^{S473} induced by Ism1 (100 nM) or insulin (100 nM) in HSMC cells at 5 min. (D) Western blot (n=2 biological replicates) of pAKT^{S473} induced by commercially available Ism1 (100 nM, R&D systems) in 3T3-F442A cells at 5 min. (E) Western blot (n=1 biological replicate at 6 time points) of signaling induced by Ism1 (200 nM) or insulin (100 nM) in 3T3-F442A cells at different time points. (F) Western blot (n=1 biological replicate per treatment) of pAKT^{S473} induced by Ism1 (100 nM) or insulin (100 nM) in mouse adipocytes at 5 min after pre-treatment with PI3K inhibitors wortmannin, LY294002, or mTORC1 inhibitor rapamycin, or Insulin/IGFR inhibitor OS1906. (G) Representative western blot (n=2 in total) of pTyr levels following stimulation with vehicle (Ctl), or 100 nM Ism1, or 100 nM insulin for 2 minutes. (H) Western blot (n=1 biological replicate per treatment) of pAKT, AKT and β-actin in 3T3-F442A cells pre-treated for 30 min with LDC1267 at indicated doses followed by treatment for 5 min with vehicle (0), Ism1 (200 nM), insulin (200 nM), or PDGF-ββ (20 ng/ml).

FIG. 12. Ism1 overexpression prevents hepatic steatosis in DIO mice (A) Ism1 gene expression in livers from AAV8-GFP and AAV8-ism1 mice (n=10 mice per group). (B-E) Ism1 gene expression in BAT (B), skeletal muscle (C), iWAT (D) and brain (E) from AAV8-GFP and AAV8-Ism1 mice (n=10 mice per group). (F) Body composition in AAV8-GFP and AAV8-Ism1 mice measured after 10 weeks of HFD (n=10 mice per group). (G) Food intake in AAV8-GFP and AAV8-Ism1 mice measured after 4 weeks of HFD (n=8 mice per group). (H) Energy expenditure in AAV8-GFP and AAV8-Ism1 mice measured after 4 weeks of HFD before any differences in body weights were observed (n=8 mice per group). (I) Plasma glucose levels in AAV8-GFP and AAV8-Ism1 mice measured after 10 weeks of HFD (n=10 mice per group). (J) Plasma glucagon levels in AAV8-GFP and AAV8-Ism1 mice measured after 10 weeks of HFD (n=10 mice per group). (K) Representative H&E staining of BAT and iWAT tissues in AAV8-GFP and AAV8-Ism1 mice (n=10 mice per group). (L) Plasma cholesterol levels in AAV8-GFP and AAV8-Ism1 mice measured after 10 weeks of HFD (n=10 mice per group). Data are presented as mean±S.E.M of biologically independent samples. *P<0.05, **P<0.01, ***P<0.001 by two tailed Student's t-test (a, b, c, d, e, f, l, j, l) or two-way Anova for repeated measurements (g, h).

FIG. 13. Ism1 suppresses de novo lipogenesis and promotes protein synthesis in hepatocytes. (A) De novo lipogenesis in mouse AML12 hepatocytes after 24 h treatment with Ism1 in the presence or absence of 200 nM insulin (n=3 biological replicates). (B) Srebp1c gene expression in primary mouse adipocytes after Ism1 or insulin treatment (n=4 biological replicates). (C) Western blot (n=1 biological replicate per treatment) of Srebp1 in mouse adipocytes after treatment with Ism1 in the presence or absence of insulin. (D-G) Srebp1c, Fas, Acc, and ChREBPP gene expression in adipocytes treated with Ism1 in the presence or absence of insulin (n=3 biological replicates). (H) ChREBPP gene expression in AML12 hepatocytes treated with Ism1 in the presence or absence of insulin (n=3 biological replicates). (I) Pgc1 β gene expression in AML12 hepatocytes treated with Ism1 in the presence or absence of insulin (n=3 biological replicates). (J) Representative western blot (n=2 biological replicates) of AKT^{S473}, AKT and β-actin in AML12 hepatocytes treated with Ism1 for 5 min in the presence or absence of insulin. Data are presented as mean±S.E.M of biologically independent samples. *P<0.05, **P<0.01, ***P<0.001 by two tailed Student's t-test (a, b, d, e, f, g, h, i).

FIG. 14. Therapeutic administration of recombinant Ism1 improves glucose tolerance and hepatic steatosis. (A) Representative western blot (n=1 biological replicate per time point) of plasma Ism1 after I.V. administration of ISM1 protein in mice. (B) Representative western blot (n=2 biological replicates) of iWAT pAKT^{S473}, AKT, and β-actin after I.V. administration of Ism1 protein in 16 weeks DIO mice. (C) Overview of therapeutic administration of Ism1 protein by daily I.P. injections of 5 mg/kg protein in 12 weeks DIO mice for 5 days. (D-G) Body weights (D), blood glucose (E), GTT (F), and ITT (G) after 5 days of daily I.P. injections of 5 mg/kg protein in 12 weeks DIO mice (n=5 mice per group) Data are presented as mean±S.E.M of biologically independent samples. *P<0.05, **P<0.01, ***P<0.001 by two tailed Student's t-test (d, e) or two-way Anova (f, g).

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “obesity-related condition” refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, type 2 diabetes, metabolic syndrome, fatty liver disease such as NASH, hyperglycemia, hyperinsulinemia, impaired glucose tolerance, impaired fasting glucose, hyperlipidemia, hypertriglyceridemia, insulin resistance, hypercholesterolemia, atherosclerosis, coronary artery disease, peripheral vascular disease, and hypertension.

Diabetes is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”).

According to recent estimates by the World Health Organization, more than 200 million people worldwide have diabetes, whereby 90% suffer from type 2 diabetes mellitus. Typical long term complications include development of neuropathy, retinopathy, nephropathy, generalized degenerative changes in large and small blood vessels and increased susceptibility to infection. Since individuals with type 2 diabetes still have a residual amount of insulin available in contrast to type 1 diabetic individuals, who completely lack the production of insulin, type 2 diabetes only surfaces gradually and is often diagnosed several years after onset, once complications have already arisen.

Insulin resistance occurs in 25% of non-diabetic, non-obese, apparently healthy individuals, and predisposes them to both diabetes and coronary artery disease. Hyperglycemia in type II diabetes is the result of both resistance to insulin in muscle and other key insulin target tissues, and decreased beta cell insulin secretion. Longitudinal studies of individuals with a strong family history of diabetes indicate that the insulin resistance precedes the secretory abnormalities. Prior to developing diabetes these individuals compensate for their insulin resistance by secreting extra insulin. Diabetes results when the compensatory hyperinsulinemia fails. The secretory deficiency of pancreatic beta cells then plays a major role in the severity of the diabetes.

Type II diabetes mellitus is characterized by insulin resistance and hyperglycemia, which in turn can cause retinopathy, nephropathy, neuropathy, or other morbidities. Additionally, diabetes is a well-known risk factor for atherosclerotic cardiovascular disease. Metabolic syndrome refers to a group of factors, including hypertension, obesity, hyperlipidemia, and insulin resistance (manifesting as frank diabetes or high fasting blood glucose or impaired glucose tolerance), that raises the risk of developing heart disease, diabetes, or other health problems; (Grundy et al, Circulation. 2004; 109:433-438).

There is a well-characterized progression from normal metabolic status to a state of impaired fasting glucose (IFG: fasting glucose levels greater than 100 mg/dL) or to a state of impaired glucose tolerance (IGT: two-hour glucose levels of 140 to 199 mg/dL after a 75 gram oral glucose challenge). Both IFG and IGT are considered pre-diabetic states, with over 50% of subjects with IFG progressing to frank type II diabetes within, on average, three years (Nichols, Diabetes Care 2007. (2): 228-233).

Non-alcoholic fatty liver disease (NAFLD) and non-alcoholic steatohepatitis (NASH) are conditions associated with fatty infiltration of the liver. NAFLD is one cause of a fatty liver, occurring when fat is deposited (steatosis) in the liver not due to excessive alcohol use (Clark J M et al, J. American Medical Association 289 (22): 3000-4, 2003). It can be related to insulin resistance and the metabolic syndrome and may respond to treatments originally developed for other insulin-resistant states (e.g. diabetes mellitus type 2) such as weight loss, metformin and thiazolidinediones.

NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. NASH is a progressive disease: over a 10-year period, up to 20% of patients with NASH will develop cirrhosis of the liver, and 10% will suffer death related to liver disease. NASH is the most extreme form of NAFLD, and is regarded as a major cause of cirrhosis of the liver of unknown cause (McCulough A J et al, Clinics in Liver Disease 8 (3): 521-33, 2004).

Common findings in NAFLD and NASH are elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver disease and can be used to assess the severity of the inflammation and resultant fibrosis (Adams L A et al, Postgrad Med J 82(967):315-22, 2006). Non-invasive diagnostic tests have been developed, such as FibroTest, that estimates liver fibrosis, and SteatoTest, that estimates steatosis, however their use has not been widely adopted (McCulough A J et al, Clinics in Liver Disease 8 (3): 521-33, 2004).

The term ISM1 agent is used to refer to ISM1 polypeptides and nucleic acids that encode them. Isthmin (ism) is a secreted protein, with 2 ISM genes in the genome of mammals, ISM1 and ISM2/Tail1, both of which encode secreted proteins that exhibit signal peptides, as well as thrombospondin (TSR) and adhesion-associated (AMOP) domains. ISM1 is located in human chromosome 20. ISM1 has been suggested to have antiangiogenic, antitumorigenic, and proapoptotic properties. The terms “Ism1 gene product”, “ISM1 polypeptide”, and “ISM1 protein” are used interchangeably herein to refer to native sequence ISM1 polypeptides, ISM1 polypeptide variants, ISM1 polypeptide fragments and chimeric ISM1 polypeptides that can modulate insulin resistance and hepatic steatosis.

Native sequence ISM1 polypeptides include the proteins ISM1, NCBI Reference Sequence: NP_543016.1, provided here as SEQ ID NO:1; particularly the mature protein, which comprises amino acid residues 20-464:

MVRLAAELLLLLGLLLLTLHITVLRGSGAADGPDAAAGNASQAQLQNNLN
VGSDTTSETSFSLSKEAPREHLDHQAAHQPFPRPRFRQETGHPSLQRDFP
RSFLLDLPNFPDLSKADINGQNPNIQVTIEVVDGPDSEADKDQHPENKPS
WSVPSPDWRAWWQRSLSLARANSGDQDYKYDSTSDDSNFLNPPRGWDHTA
PGHRTFETKDQPEYDSTDGEGDWSLWSVCSVTCGNGNQKRTRSCGYACTA
TESRTCDRPNCPGIEDTFRTAATEVSLLAGSEEFNATKLFEVDTDSCERW
MSCKSEFLKKYMHKVMNDLPSCPCSYPTEVAYSTADIFDRIKRKDFRWKD
ASGPKEKLEIYKPTARYCIRSMLSLESTTLAAQHCCYGDNMQLITRGKGA
GTPNLISTEFSAELHYKVDVLPWIICKGDWSRYNEARPPNNGQKCTESPS
DEDYIKQFQEAREY

ISM1 polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as a therapeutic agents in the methods disclosed herein, as do nucleic acids encoding these polypeptides or their transcriptionally active domains and vectors comprising these nucleic acids. In certain embodiments, the ISM1 agent is human ISM1 protein.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. Asparagine (N)-linked glycosylation is an important post-translational modification that results in the covalent attachment of oligosaccharides onto asparagine residues in a protein sequence. The native ISM1 protein is N-glycosylated. For therapeutic purposes the protein may have the native glysylation pattern, may have a modified glycosylation pattern or may be aglycosylated.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound. Proteins may be recombinantly produced.

In some embodiments, the ISM1 protein can be conjugated to additional molecules to provide desired pharmacological properties such as extended half-life. In one embodiment, ISM1 protein is fused to the Fc domain of IgG, albumin, or other molecules to extend its half-life, e.g. by pegylation, glycosylation, and the like as known in the art. In some embodiments the ISM1 protein is conjugated to a polyethylene glycol molecules or “PEGylated.” The molecular weight of the PEG can include but is not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the polypeptide or via a linker molecule. The processes and chemical reactions necessary to achieve PEGylation of biological compounds is well known in the art.

ISM1 protein can be acetylated at the N-terminus, using methods known in the art, e.g. by enzymatic reaction with N-terminal acetyltransferase and, for example, acetyl CoA. ISM1 protein can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The ISM1 protein be fused to the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, ISM1 protein can comprise a polypeptide that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In some embodiments, the chimeric polypeptide further comprises a C-terminal c-myc epitope tag.

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidermal, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for co-administration with the active agents of the invention can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

In some embodiments a unit dose is at least about 0.1 mg/kg, at least about 0.5 mg/kg, at least about 1 mg/kg, at least about 5 mg/kg, at least about 10 mg/kg, at least about 20 mg/kg, at least about 50 mg/kg, at least about 100 mg/kg, in some embodiments the effective dose is from about 1 to 50 mg/kg. Dosing may be daily, every 2 days, every 3 or more days, e.g. weekly, semi-weekly, bi-weekly, monthly, etc. Dosing may be parenteral, including sustained release formulations. Dosing may be maintained for long periods of time, e.g. months, or years, to maintain desirable glucose and fatty acid levels.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the active agent may be administered in dosages of 0.01 mg to 500 mg/kg body weight per day, e.g. about 20 mg/day for an average person. Dosages will be appropriately adjusted for pediatric formulation.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of fatty liver disease in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Efficacy of treatment can be monitored with various methods known to the art, to determine an improvement in insulin sensitivity or liver function (or decrease in insulin resistance), where an improvements may be, for example, 5%, 20%, 15%, 20%, 25%, 30%, 40%, 50% or more improvement. Hyperinsulinemic euglycemic clamp (HEC) is known to be the “gold standard” for the measurement of insulin sensitivity. However simplified assays can be used in quantification of insulin sensitivity. There are two major groups of insulin sensitivity indices: (1) Indices calculated by using fasting plasma concentrations of insulin, glucose and triglycerides, (2) indices calculated by using plasma concentrations of insulin and glucose obtained during 120 min of a standard (75 g glucose) OGTT. The former group include homeostasis model assessment-insulin resistance (HOMA-IR), QUIKI INDEX, and McAuley index while latter include, Matsuda, Belfiore, Cederholm, Avignon and Stumvoll index. For clinical uses HOMA-IR, QUIKI, and Matsuda are suitable while HES, McAuley, Belfiore, Cederholm, Avignon and Stumvoll index are suitable for epidemiological/research purposes.

The HEC-derived index of insulin sensitivity (ISI_{HEC}, ml/kg/min/μlU ml) is obtained during a steady state period of HEC. ISI_{HEC}=MCR/Iₘₑₐₙ where, Iₘₑₐₙ−average steady state plasma insulin response (plU/ml), MCR: Metabolic clearance rate of glucose (ml/kg/min). MCR=Mₘₑₐₙ/(Gₘₑₐₙ×0.18), where Mₘₑₐₙ: Metabolized glucose expressed as average steady state glucose infusion rate per kg of body weight (mg/kg/min) Gₘₑₐₙ:Average steady state blood glucose concentration (mmol/I) 0.18-conversion factor to transform blood glucose concentration from mmol/I into mg/ml.

HOMA is a model of the relationship of glucose and insulin dynamics that predicts fasting steady-state glucose and insulin concentrations for a wide range of possible combinations of insulin resistance and β-cell function. The HOMA model has proved to be a robust clinical and epidemiological tool for the assessment of insulin resistance, where IR_{HOMA}=(I₀×G₀)/22.5(mathematically:e^{-Inx}=1/x).

Quantitative insulin sensitivity check index (QUICKI) is an empirically-derived mathematical transformation of fasting blood glucose and plasma insulin concentrations that provide a consistent and precise ISI with a better positive predictive power. It is a variation of HOMA equations, as it transforms the data by taking both the logarithm and the reciprocal of the glucose-insulin product, thus slightly skewing the distribution of fasting insulin values. It employs the use of fasting values of insulin and glucose as in HOMA calculations. QUICKI is virtually identical to the simple equation form of the HOMA model in all aspects, except that a log transform of the insulin glucose product is employed to calculate QUICKI. The QUICKI can be determined from fasting plasma glucose (mg/dl) and insulin (plU/ml) concentrations.

McAuley index is used for predicting insulin resistance in normoglycemic individuals. Regression analysis was used to estimate the cut-off points and the importance of various data for insulin resistance (fasting concentrations of insulin, triglycerides, aspartate aminotransferase, basal metabolic rate (BMI), waist circumference). A bootstrap procedure was used to find an index most strongly correlating with insulin sensitivity index, corrected for fat-free mass obtained by HEC (Mffm/I).

Matsuda index derives an ISI from the OGTT. In these methods, the ratio of plasma glucose to insulin concentration during the OGTT is used. The OGTT ISI (composite) is calculated using both the data of the entire 3 h OGTT and the first 2 h of the test. The composite whole-body insulin sensitivity index (WBISI) is based on insulin values given in microunits per milliliter (pU/mL) and those of glucose, in milligrams per deciliter (mg/L) obtained from the OGTT and the corresponding fasting values.

Liver function tests including, alkaline phosphatase (ALP), serum albumin, aspartate aminotransferase (AST), bilirubin, alanine aminotransferase (ALT), platelet count, international normalized ratio (INR), total cholesterol (Chol), triglycerides (TGs), high-density lipoprotein (HDL) cholesterol, low-density lipoprotein (LDL) cholesterol, and hemoglobin A1c (HbA1c). Fatty liver or steatosis, is considered when more than 5% of the hepatocytes present ectopic lipid droplets in a liver biopsy. Steatohepatitis requires the presence of lobular inflammation and hepatocyte lesion, usually in the form of hepatocellular ballooning with or without Mallory-Denk bodies, besides steatosis. The gold standard for the diagnosis is the liver biopsy, mainly for diagnosing NASH and staging fibrosis. However, for the diagnosis and quantification of steatosis, H¹-MRS is a reference that assesses larger volumes of liver and it detects amounts of triglycerides that may not be enough to form macrovesicles amenable of histological visualization. A histological score, NAS, combines different degrees of steatosis, hepatocellular ballooning and lobular inflammation was designed to help monitoring the effect of interventional and therapeutic strategies. Steatosis activity fibrosis (SAF) score sequentially adds steatosis, ballooning and lobular inflammation for the diagnosis of NASH. Abdominal ultrasound can be used for the diagnosis of hepatic steatosis, with 60%-94% sensitivity and 66%-97% specificity. Magnetic resonance is more sensitive than ultrasound, mainly for the diagnosis of mild steatosis. There are several combined panels with clinical and laboratorial data that were designed to predict steatosis.

Regarding estimation of liver fibrosis, NAFLD Fibrosis Score (among other non-invasive scores) has been shown to be useful in predicting overall and liver-related mortality, in 3 studies with follow up ranging from 8 to 14 years. Transient elastography (Fibroscan®), also has good accuracy to predict advanced fibrosis and exclude it in NAFLD, with cutoffs higher and lower than 9.6 and 7.9, respectively.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

With the increasing prevalence of type 2 diabetes and fatty liver disease, there is an unmet need to better treat hyperglycemia and hyperlipidemia simultaneously. Here, we identify a dual role for the adipokine Isthmin-1 (ISM1) in increasing adipose glucose uptake while suppressing hepatic lipid synthesis. ISM1 ablation results in impaired glucose tolerance, reduced adipose glucose uptake and reduced insulin sensitivity, demonstrating an endogenous function for ISM1 in glucose regulation. Mechanistically, ISM1 activates a PI3K/AKT signaling pathway independently of the insulin receptors. Notably, while the glucoregulatory function is shared with insulin, ISM1 counteracts lipid accumulation in the liver by switching hepatocytes from a lipogenic to a protein synthesis state. Furthermore, therapeutic dosing of recombinant ISM1 improves diabetes in diet-induced obese mice and ameliorates hepatic steatosis in a diet-induced fatty liver mouse model. These findings uncover an unexpected, bioactive ligand that has therapeutic activity for diabetes and fatty liver disease.

Ism1 was first identified as a gene expressed in the Xenopus midbrain-hindbrain organizer called Isthmus, with a proposed role during early brain development. The Ism1 gene is conserved in mice and humans, but the function in adult physiology has remained elusive. We show with genetic models and pharmacological approaches that ISM1 is a signaling polypeptide factor that regulates glucose uptake while suppressing lipid accumulation. Therefore, ISM1 is a biologically bioactive ligand that dissociates glucose uptake from lipid synthesis, providing new insights into metabolic regulation and offering new therapeutic avenues for glucose and lipid-associated disorders.

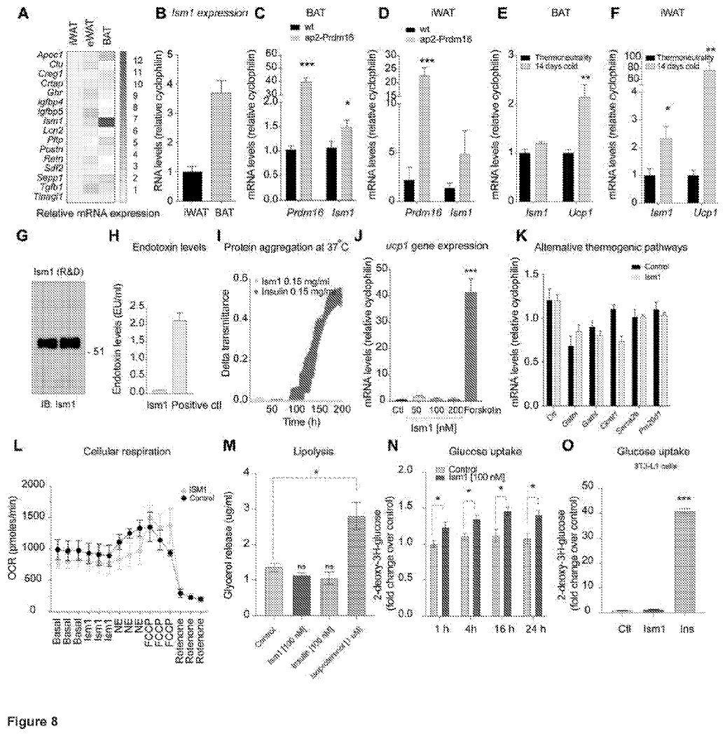

ISM1 is a secreted protein that induces glucose uptake in human and mouse adipocytes. Activation of thermogenic adipose tissue is associated with improved metabolic health, but the secreted factors from thermogenic adipocytes remain understudied. To identify adipose-derived proteins with hormone-like properties, we combined bioinformatic analyses with expression data on mature brown and white adipocytes. First, we utilized an RNA sequencing dataset from mature adipocytes isolated from murine inguinal (iWAT), epididymal (eWAT), and brown adipocytes (BAT) using ucp1-TRAP mice and applying the secreted prediction software SignalP. This filter generated 512 predicted secreted proteins. Second, to capture only those proteins that were bona fide secreted factors via classical secretion, we screened these genes against peptides detected from the TMT multiplexed proteomic secretome from cultured adipocytes derived from “beiged” ap2-PRDM16 mice. This generated a list of 16 proteins that we classify as “hormone-like” (FIG. 8A).

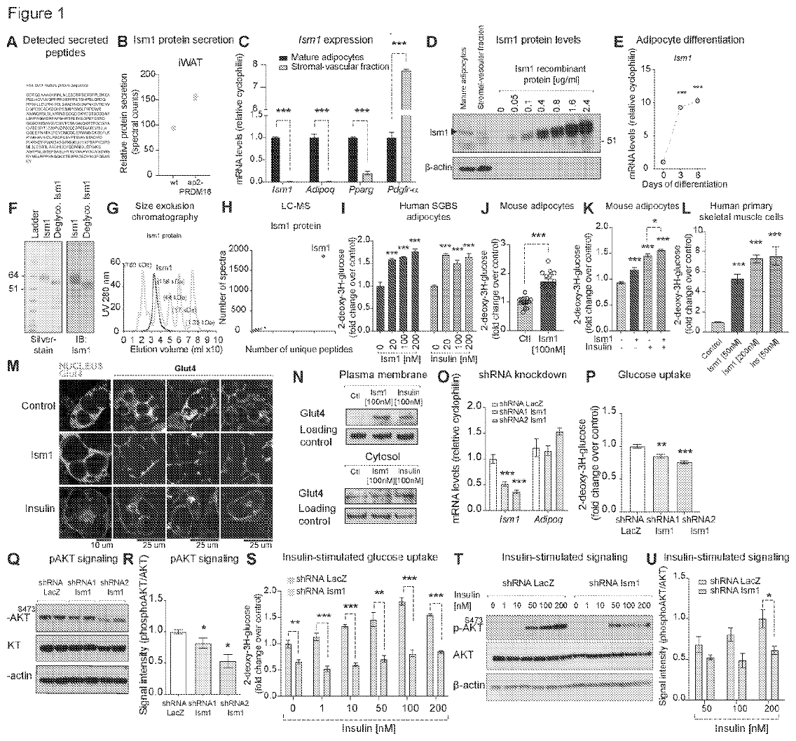

Two independent peptides were found specific to the protein ISM1 in secreted medium from cultured adipocytes, confirming the presence and identity of the mature protein as a secreted molecule with an average peptide intensity of 150 in adipocytes (FIG. 1A-B). Ism1 is also enriched in mature brown fat cells compared to iWAT and eWAT by RNA sequencing of the adiponectin-TRAP mice (FIG. 8A) and has higher expression in BAT relative to iWAT (FIG. 8B).

Furthermore, Ism1 expression is higher in ap2-prdml6 “beiged” inguinal white fat (FIG. 8C-D) and is induced in iWAT upon cold exposure (FIG. 8E-F). Importantly, RNA and protein analyses of isolated mature adipocytes demonstrate that ISM1 is almost exclusively expressed in mature fat cells (FIG. 1C-D), while negligible Ism1 expression is seen in the stromal vascular fractions from the adipose tissue.

The shared expression signatures between Ism1, Adiponectin, and Pparg (FIG. 1C) are also evident by the robust increase of Ism1 during differentiation (FIG. 1E). Besides adipose tissues, Ism1 is also expressed in other cell types such as in the skin, in mucosal tissues, and in immune cells. Here we observe robust expression of ISM1 in mature fat cells, but no function in adipose tissue or in metabolism has previously been described.

To test ISM1's possible metabolic functions, we generated recombinant mouse ISM1 protein with a C-terminal myc-his tag in mammalian HEK 293 Expi cells followed by a His trap FF column purification. The protein underwent buffer exchange and was stored in PBS −80° C. at a concentration of >1 mg/ml. SDS-gel electrophoresis analysis demonstrates a single purified mature protein around 60-65 kDa under reducing conditions (FIG. 1F), similar to commercially available recombinant mouse ISM1 protein (FIG. 8G). The identity of the protein is also validated using an ISM1 antibody (FIG. 1F). Treatment with deglycosylating enzymes, including PNGase F demonstrates a band shift from 65 kDa to 60 kDa, suggesting that ISM1 is N-glycosylated (FIG. 1F). Glycan analysis by LC-MS/MS confirms the presence of several N-linked glycan masses at the N-terminus of the ISM1 protein and reveals novel glycosylation and phosphorylation sites on ISM1. Size exclusion chromatography fractionation under native conditions shows that the ISM1 protein elutes as a single peak, but likely forms dimers or oligomers (FIG. 1G). Importantly, the ISM1 protein is highly pure and devoid of other protein contaminants, as demonstrated by global protein analysis by LC-MS/MS (FIG. 1H). Approximately 1800 peptides for ISM1 are found in the purified ISM1 protein preparations, while <2 peptides are found for ligands such as platelet-derived growth factor, epidermal growth factor, and insulin, demonstrating the purity of the protein. Importantly for in vivo work, the protein was tested for the presence of endotoxin with levels below 0.1 EU/ml (FIG. 8H). There is no evidence of compromised stability when the protein is incubated at 370C for up to 200 hours, as shown by a protein aggregation assay measuring increasing transmittance as a function of time (FIG. 8I). Insulin, well known to form aggregated fibrils, is used as a positive control. The yield for a representative preparation of recombinant ISM1 protein is consistently around 50 mg/L. These data demonstrate the efficient production of highly pure recombinant ISM1 protein for in vitro and in vivo studies.

Adipose tissue plays a role in glucose regulation by accounting for 10-15% of the total glucose uptake. Therefore, we next asked whether ISM1 increases glucose uptake in adipocytes. Fully differentiated human SGBS adipocytes were treated with ISM1 or insulin at doses ranging from 10 nM to 200 nM before assaying for glucose uptake using [³H]-2-deoxy-glucose. As expected, insulin induces an increase in glucose uptake in SGBS cells starting from 20 nM (FIG. 1I). Interestingly, ISM1 also induces a 1.5-fold increase in glucose uptake starting from 20 nM, as robustly as insulin in SGBS cells (FIG. 1I). In addition to human SGBS adipocytes, 100 nM ISM1 increases glucose uptake in primary mouse adipocytes, consistently around 1.5-fold across multiple experiments (FIG. 1J). Importantly, the effect of ISM1 is seen in the absence of insulin. Expectedly, insulin induced a higher glucose induction than ISM1, but the addition of 100 nM ISM1 in cells treated with 50 nM insulin further increased insulin-induced uptake in adipocytes (FIG. 1K). In addition, prolonged ISM1 treatment does not lead to desensitization, as a 24 h protein treatment induced a similar degree of glucose uptake induction as 4 h (FIG. 8N). Importantly, 100 nM albumin used as a protein control had no effect on glucose uptake, demonstrating the specific bioactivities of ISM1 (FIG. 8N).

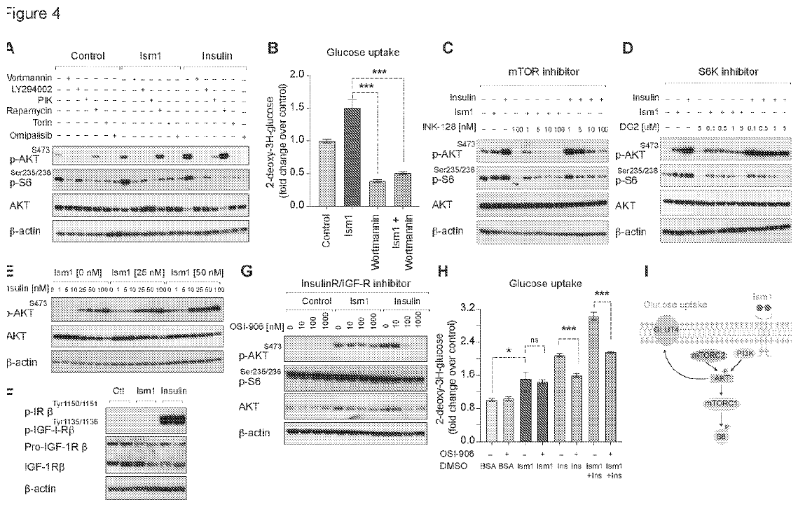

The induction of glucose uptake is not restricted to adipocytes, as experiments in human primary skeletal muscle cells showed a 5-fold induction of glucose uptake at 50 nM ISM1 and 7-fold at 200 nM ISM1 compared with control cells (FIG. 1L). Surprisingly, ISM1 does not induce glucose uptake in differentiated 3T3-L1 adipocytes, while insulin induces an almost 40-fold induction in this cell line, suggesting cell type-specific receptors or glucose transport for ISM1 (FIG. 8O). In conclusion, the cellular studies using four different cell types show that ISM1 induces glucose uptake by 1.25-fold up to 6-fold. The effects of ISM1 are <50% of that of insulin in primary mouse adipocytes (FIG. 1K), 50-100% in SGBS cells (FIG. 1I and FIG. 4H), and 70% in human skeletal muscle cells (FIG. 1L). These results show that the effects on ISM1-induced glucose transport are cell-type dependent and that ISM1 might act on a low-abundant cell surface receptor.

GLUT4 is the predominant insulin-sensitive transporter in adipose tissue and is, in unstimulated conditions, compartmentalized in intracellular vesicles. Upon insulin stimulation in insulin-sensitive tissues, GLUT4 is translocated to the cell surface to increase glucose import. To determine if ISM1 promotes translocation of GLUT4 to the plasma membrane, we treated cells with 100 nM insulin or ISM1, followed by GLUT4 immunostaining and analysis using confocal microscopy. Both ISM1 and insulin treatment induce higher levels of GLUT4 at cell surface compared with control cells, which demonstrated almost exclusively intracellular compartmentalized GLUT4 (FIG. 1M). Biochemical fractionation to separate the plasma membrane from cytosolic fractions confirms the translocation of GLUT4 to the plasma membrane after stimulation with both ISM1 and insulin, but not with control treatment. Expectedly, the membrane-bound receptor PDGFR-α used as a loading control is enriched in the plasma membrane fraction and does not change with either of the treatments (FIG. 1N). These results show that ISM1 increases adipocyte glucose uptake at least in part by translocating GLUT4 to the cell surface.

Intrigued by the exogenous action of ISM1 in regulating glucose transport, we next wanted to determine if endogenous ISM1 is sufficient to control glucose uptake. To this end, we generated two shRNAs in adenoviral vectors against mouse Ism1 to reduce its expression; we then assessed the effects of acute Ism1 knockdown on glucose uptake. The Ism1 shRNAs generates a 50% and 70% knockdown efficiency without affecting differentiation as determined by no change in adiponectin expression (FIG. 1O). Interestingly, Ism1 knockdown prevents glucose uptake in adipocytes, demonstrating that the endogenous levels of ISM1 contribute to basal glucose uptake in adipocytes (FIG. 1P). Because glucose uptake involves activation of the regulatory subunit of type 1A phosphatidylinositol 3-kinase (PI3K) and phosphorylation of AKT at S473, we next evaluated the levels of pAKT^{S473} in ISM1 knockdown cells. We observe a 20-50% reduction in pAKT signaling in ISM1-shRNA adipocytes compared with LacZ-shRNA, strongly indicating that endogenous ISM1 is necessary to maintain the AKT basal signaling tone (FIG. 1Q-R). Interestingly, ISM1 is also important for insulin-dependent glucose uptake; cells with reduced ISM1 levels completely fail to respond to insulin-induced glucose uptake even at maximal insulin concentrations (FIG. 1S) and have reduced pAKT signaling compared with control cells (FIG. 1T-U). Taken together, our data identify ISM1 as a secreted factor that exogenously and endogenously controls glucose uptake in vitro.

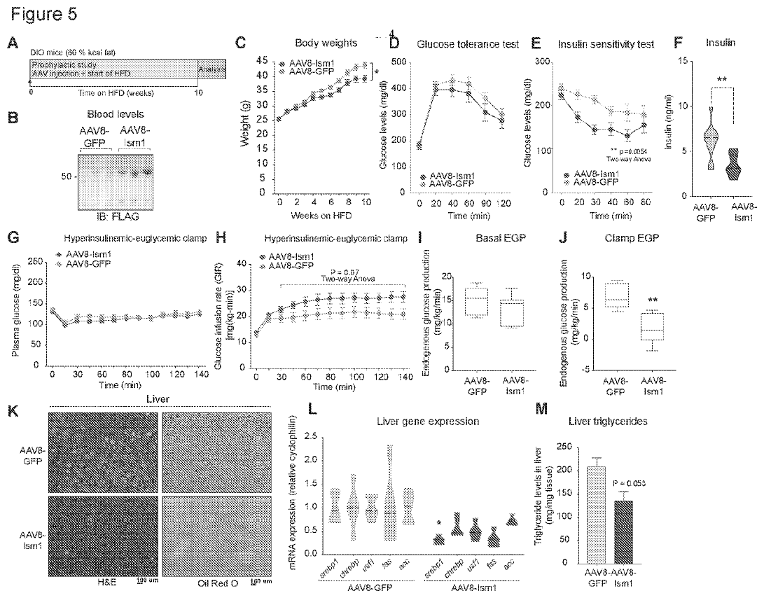

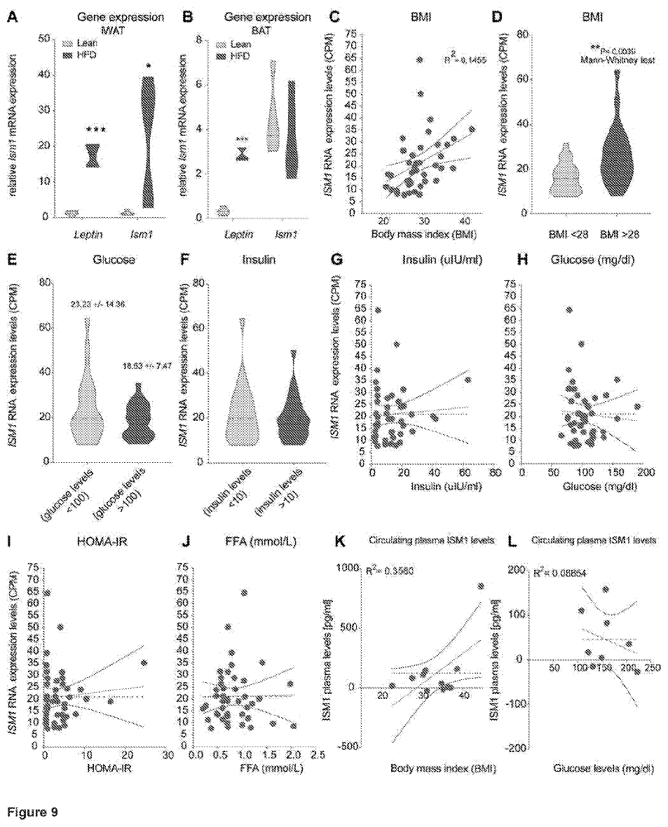

ISM1 expression is correlated with obesity in mice and humans. Many metabolic hormonal factors are elevated in individuals with metabolic dysfunction, including insulin, FGF21, FGF1, and GDF15. Therefore, we asked whether ISM1 levels are changed by nutritional and obesity status. To investigate this, diet-induced obese mice were fed a high-fat diet for 16 weeks. As predicted, leptin levels are increased almost 20-fold in iWAT (FIG. 9A) and 5-fold in BAT (FIG. 9B), compared with lean mice. Interestingly, Ism1 gene expression is on average increased 30-fold higher in iWAT but not different in BAT (FIG. 9A-B). To examine whether Ism1 expression was regulated in obese adipose tissue in humans, we collected human subcutaneous mature adipocytes and performed RNA sequencing from 43 individuals with known clinical metabolic parameters. In this dataset, Ism1 expression is positively and significantly correlated with body mass index (BMI) (FIG. 9C). When stratifying individuals based on parameters for weight, insulin, or glucose levels, Ism1 expression was higher in individuals with a BMI >28 (FIG. 9D). On the contrary, Ism1 transcript levels do not significantly correlate with glucose, insulin, HOMA-IR, or free fatty acid levels in these individuals (FIG. 9E-J). Importantly, by generating a monoclonal ISM1 antibody, we observe that circulating plasma levels of human ISM1 is detected at an average of 50 pg/ml, and trend to positively correlate with BMI (FIG. 9K) but not with glucose (FIG. 9L) in female individuals. These results suggest that ISM1 is a bona fide hormone and that circulating levels are physiologically regulated by nutritional and metabolic changes in mice and humans.

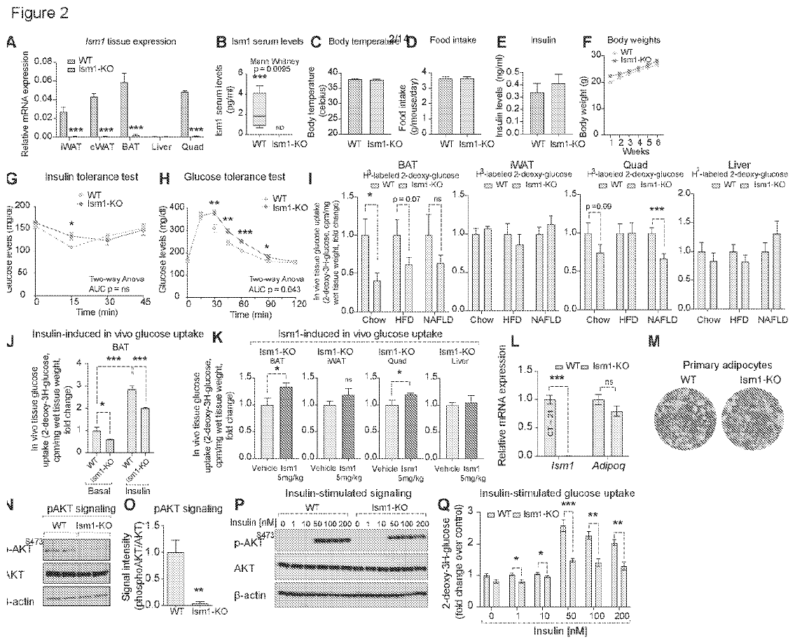

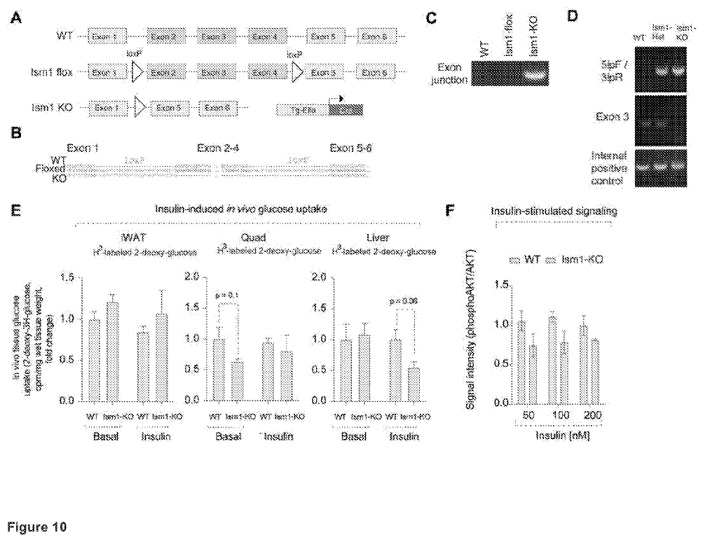

Ablation of ISM1 causes glucose intolerance and impaired insulin-stimulated adipocyte glucose uptake. Based on the findings that ISM1 exogenously and endogenously controls glucose uptake in vitro, we next sought to investigate the endogenous function of ISM1 in physiology by analyzing mice with a targeted deletion of the Ism1 gene. First, we generated the Ism1 floxed allele spanning exons 2-4 by using CRISPR-mediated gene editing. Second, the Ism1 floxed allele was crossed with female mice expressing the Ella-Cre transgene for embryonic deletion (FIG. 10A-B). The genomic exon deletion was confirmed with pcr amplifying the exon junction as well as Sanger sequencing (FIG. 10B-C). The ISM1-flox heterozygote mice were mated to generate germline deletion of ISM1, which was confirmed with pcr genotyping of the wildtype (WT), heterozygotes (Het) and knockout (ISM1-KO) mice (FIG. 10D). The loss of Ism1 mRNA expression in iWAT, eWAT and BAT tissue in ISM1-KO mice compared with WT mice is also confirmed by qPCR (FIG. 2A). We find that ISM1 circulates at 2-4 pg/ml in mouse serum, and expectedly, is not detected in the ISM1-KO mice (FIG. 2B). Interestingly, while body temperature (FIG. 2C), food intake (FIG. 2D), insulin levels (FIG. 2E), and body weights (FIG. 2F) are indistinguishable between WT and ISM1-KO littermates under chow diet, there is a trend towards higher insulin resistance in the ISM1-KO mice (FIG. 2G) and significantly worsened glucose tolerance (FIG. 2H). These results suggest that ISM1 regulates peripheral glucose uptake.

To directly test the hypothesis that ISM1 ablation results in reduced tissue glucose uptake, we performed radiolabeled tracing using [³H]-2-deoxy-glucose in WT or ISM1-KO mice under three dietary conditions; chow, HFD, or NAFLD for four weeks prior to the measurements. Interestingly, we observe reduced BAT glucose uptake in the ISM1-KO mice under all three diets, but the difference is only significant under chow conditions. Skeletal muscle from ISM1-KO mice also has a reduced glucose uptake under NAFLD conditions, while iWAT and liver show no differences between the genotypes (FIG. 2I). Furthermore, analysis of basal and insulin-induced tissue-specific glucose uptake showed that BAT has the largest defect in glucose uptake under both basal and insulin-stimulated conditions in the ISM1-KO mice compared with WT mice (FIG. 2J and FIG. 10E).

Based on these data, we conclude that the reduced glucose tolerance in the ISM1-KO mice is likely due to a combination of an impaired basal glucose uptake in BAT and skeletal muscle and reduced insulin sensitivity in BAT. Importantly, injection of recombinant ISM1 protein into ISM1-KO mice can increase glucose uptake in BAT and skeletal muscle (FIG. 2K), indicating that the glucose uptake phenotype is directly caused by the ISM1 deficiency rather than a secondary consequence of ISM1 loss. ISM1 protein treatment results in a trending increase in iWAT, but no hepatic glucose uptake induction is seen after ISM1 treatment. In conclusion, these data support the notion that BAT and skeletal muscle are the major tissues responsible for ISM1-mediated glucose uptake.

To directly determine the requirement for ISM1 on adipocyte glucose uptake, we isolated primary mouse inguinal cells from WT and ISM1-KO mice for in vitro differentiation into fat cells. There is no significant difference in differentiation capacity between WT and ISM1-KO cells, as determined by adiponectin expression levels (FIG. 2L) and Oil Red O staining (FIG. 2M). As expected, endogenous Ism1 is abundant in primary adipocytes, with a CT value of ˜21, while Ism1 is absent in ISM1-KO cells with a CT value of ˜31 (FIG. 2L). We observed considerably lower pAKT levels in ISM1-KO adipocytes compared with WT cells, further supporting that endogenous levels of adipocyte-secreted ISM1 contribute to the basal signaling tone (FIG. 2N-0). Lastly, ablation of ISM1 results in reduced insulin-induced signaling (FIG. 2P and FIG. 10F) and reduced insulin-induced glucose uptake (FIG. 2Q). Based on these orthogonal results from assessing short-term and long-term effects, we conclude that ISM1 is both required and sufficient for a fraction of the peripheral glucose regulation in adipocytes and whole-body glucose uptake in mice.

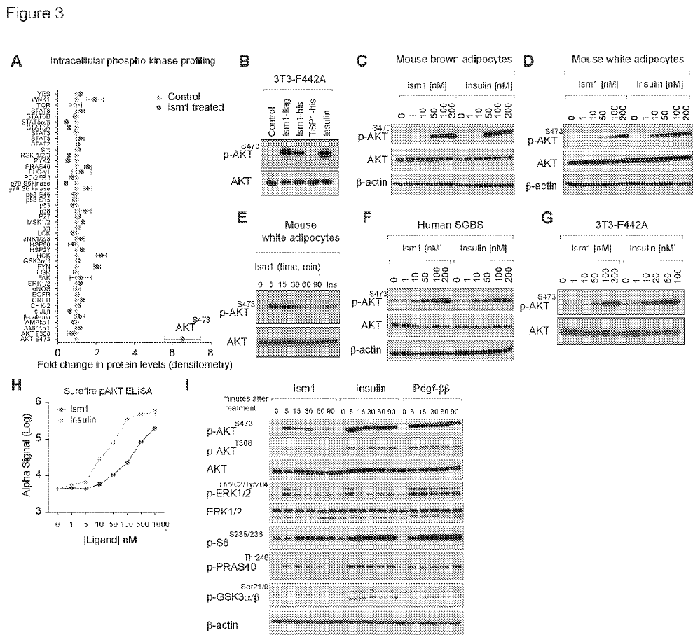

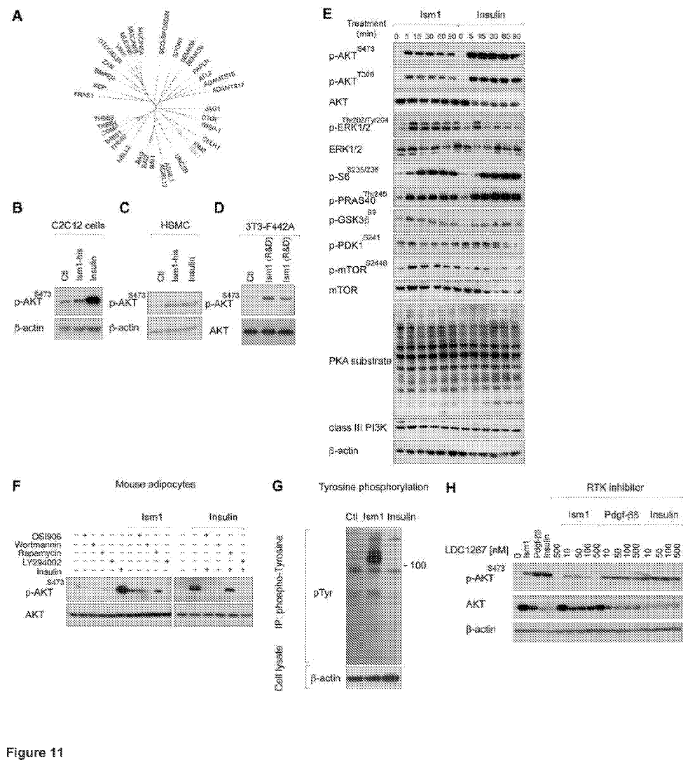

ISM1 activates the PI3K/AKT pathway. Since many hormones and growth factors are high-affinity ligands for signaling cell surface receptors, we next aimed to identify the intracellular signaling pathways involved in ISM1 function. Gene family tree analysis suggests that ISM1 is a distant relative to other proteins containing a Thrombospondin Type 1 (TSP1) domain or an Adhesion-associated domain (AMOP) (FIG. 11A). However, neither of the domains are known to possess any direct signaling activity. We first performed a phosphokinase array that detects phosphorylated levels of 43 distinct proteins simultaneously in a single sample. 3T3-F442A cells were treated for 5 min with either vehicle or ISM1 protein followed by phosphoprotein analysis. Interestingly, the top hit from this screen in response to ISM1 treatment is phosphorylated protein kinase B (PKB)/AKT at S473 (FIG. 3A). To confirm the specificity of the protein array results, and that the AKT signaling response is specific to ISM1 (and not dependent on the C-terminal tag of the protein), we performed the same experiment using a phospho-specific antibody for AKT^{S473} with ISM1 containing either a C-terminal flag tag or a C-terminal his tag. As a further control, another protein containing thrombospondin domains, a his-tagged mouse Thrombospondin-1 was used (FIG. 3B). Insulin, as well as both ISM1 proteins, show similar bioactivities on pAKT^{S473}, while Thrombospondin-1 shows no bioactivity in this assay (FIG. 3B). In addition to 3T3-F442A, ISM1 also induced pAKT^{S473} in differentiated primary mouse brown adipocytes (FIG. 3C), white adipocytes (FIG. 3D-E), human SGBS adipocytes (FIG. 3F), C2C12 skeletal muscle cells (FIG. 11B), and human primary skeletal muscle cells (HSMC) (FIG. 11C), consistent with the function of ISM1 in inducing glucose uptake in adipocytes and in skeletal muscle cells. The ISM1 protein has comparable bioactivities to commercially available ISM1 protein purchased from R&D systems (FIG. 11D). To determine the minimal dose required to induce pAKT^{S473} signaling, we treated 3T3-F442A cells with increasing ISM1 protein or insulin doses. This demonstrates a dose-dependent increase in phosphorylation starting at 50 nM for ISM1 and 10 nM for insulin (FIG. 3G). We could confirm the ISM1 bioactivity using the more quantitative AlphaLisa assay for pAKT^{S473}, showing that 100 nM ISM1 induces a similar response as 10 nM insulin (FIG. 3H).

Both mouse and human studies show that activation of PI3K and AKT in response to growth factors plays a central role in controlling metabolism. Insulin induces phosphorylation of pAKT^{S473} and pAKT^{T308}, both of which have been mechanistically linked to glucose uptake in various tissues. To evaluate the temporal signaling in response to ISM1, we performed a time-course experiment of ISM1, insulin or platelet-derived growth factor-ββ (PDGF-ββ). In addition to the phosphorylation of pAKT^{S473}, we also observed phosphorylation at pAKT^{T308} at all time points in cells treated with 100 nM ISM1 or insulin (FIG. 3I). Additionally, there was also a time-dependent activation of S6^{S235/S236} and the AKT substrate PRAS40^{Thr246}, known targets of insulin. Notably, at 100 nM, ISM1 does not evoke as robust ERK1/2 phosphorylation as the known mitogen PDGF-ββ. However, at 200 nM, ISM1 shows prolonged and more potent phosphorylation of ERK1/2 than insulin, which suggest that ISM1 and insulin induce distinct and divergent signaling responses and downstream pathways (FIG. 11E). Moreover, ISM1 does not display any activity or displays only weak activity on other pathways such as protein kinase A (PKA), PDK1 or GSK3P even at 200 nM doses (FIG. 11E). To our knowledge, this is the first robust and direct signaling action identified for ISM1.

To further interrogate the requirements for the ISM1 signaling pathway in detail, we treated cells with four inhibitors targeting the PI3K pathway: Wortmannin, LY294002, PIK or the dual PI3K/mTOR inhibitor Omipalisib. All four inhibitors completely block pAKT^{S473} phosphorylation induced by ISM1 and insulin (FIG. 4A). This effect also holds true in adipocytes, as LY294002 and Wortmannin inhibit the ISM1-induced pAKT^{S473} activity in primary adipocytes (FIG. 11F). We further show that PI3K is required for glucose regulation in adipocytes, as demonstrated by the complete blockade of ISM1-induced glucose uptake in the presence of Wortmannin (FIG. 4B). Insulin signaling components such as insulin receptor substrate (IRS-1) and IGF1R/IR are tightly controlled by the multiprotein complexes of mammalian target of rapamycin (mTOR) mTORC1 and mTORC2, and both mTOR complexes are activated by insulin. To investigate whether any of the mTORC1 complexes are involved in ISM1 signaling, cells were treated with the mTORC1 inhibitor Rapamycin or the mTORC2 inhibitor Torin, prior to ISM1 or insulin stimulation. Neither ligand activity is inhibited by Rapamycin, while Torin fully ablates the ISM1 induced AKT signaling (FIG. 4A). Similar effects are seen with the potent and selective dual mTORC1 and mTORC2 competitive inhibitor INK-128 (FIG. 4C). This suggests that mTORC1 is downstream of AKT or not involved in ISM1 or insulin signaling, while mTORC2 is upstream of AKT and is required for induction of the signaling cascade both by ISM1 and insulin. Notably, in the presence of all four mTOR inhibitors, S6^{S235/S236} phosphorylation by ISM1 is completely inhibited, suggesting that the activation of S6 is downstream of the mTOR complexes (FIG. 4A, FIG. 4C). This is also confirmed by using the S6K1 kinase inhibitor DG2, which dose-dependently inhibited S6^{S235/S236} while pAKT remained intact. These results are supporting the finding that ISM1-induced S6^{S235/S236} activation is downstream of AKT (FIG. 4D). Taken together, these data strongly suggest that ISM1 signaling shares common downstream signaling targets with insulin and requires mTORC2 involvement to induce the PI3K/AKT pathway and glucose uptake.

ISM1 signaling is independent of the Insulin- and IGF Receptors. Because the ISM1-induced signaling pathway resembled that of insulin, we next asked whether ISM1 directly engages the insulin receptors, or acutely sensitizes cells to insulin. To test this hypothesis, we treated 3T3-F442A cells with insulin using doses from 1 nM up to 100 nM in the presence or absence of 25 nM or 50 nM ISM1. We observed an additional effect of ISM1 over insulin alone on AKT activity, but no evidence of potentiation was seen, suggesting that ISM1 does not modulate the insulin-insulin receptor interaction (FIG. 4E). To assess whether ISM1 could induce insulin receptor phosphorylation, we next tested the response to ISM1 in activating IGF-I-Receptor β phosphorylation at Tyr1135/1136 or Insulin receptor at Tyr1150/1151—two well-described phosphorylation sites induced by insulin. 3T3-F442A cells were treated with either vehicle, 100 nM ISM1 or 100 nM insulin for 2 minutes followed by western blot analysis. As expected, insulin induced IGF-R/IR phosphorylation; however, no phosphorylation was seen in the ISM1 treated cells (FIG. 4F).

To address whether the presence of intact insulin receptors is required for ISM1-induced pAKT signaling, we made use of the dual receptor tyrosine kinase inhibitor OSI-906 to specifically target the insulin receptor and insulin growth factor receptor. Insulin signaling is completely abolished in the presence of 1000 nM OSI-906 as expected. However, ISM1 signaling is intact in the presence of the IGF/IR inhibitor, even at 1000 nM OSI-906 (FIG. 4G). Similarly, insulin-induced glucose uptake is significantly reduced with OSI-906, while ISM1-induced glucose uptake is not affected (FIG. 4H). Intriguingly, we find that a 2-minute ISM1 treatment induces phosphorylation of tyrosine residues on proteins >100 kDa in size, while insulin induces phosphorylation of its receptors at larger molecular weights (FIG. 11G). Moreover, we find that the ISM1-induced pAKT signaling in 3T3-F442A cells can be inhibited by an RTK inhibitor, LDC1267, which targets multiple receptor tyrosine kinases. Notably, neither the signaling induced by PDGFββ or insulin are inhibited by LDC1267 at any of the doses tested, suggesting a distinct receptor for ISM1 (FIG. 11H). In conclusion, these results strongly suggest that ISM1 signaling does not need or involve insulin receptors, but rather activates the PI3K/AKT pathway via a distinct receptor that shares a common downstream signaling signature with insulin (FIG. 4I).

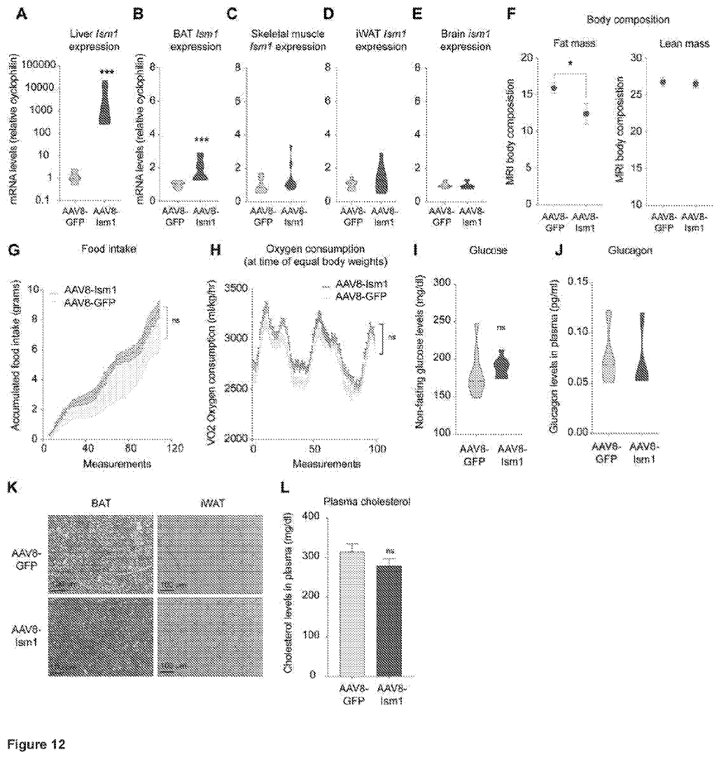

ISM1 overexpression prevents insulin resistance and hepatic steatosis in DIO mice. To study the effect of chronically elevated circulating ISM1 in vivo, we transduced mice with viral expression vectors to robustly increase circulating ISM1 levels, followed by high fat diet-feeding (FIG. 5A). 6-8-week-old mice were I.V. injected with 10¹⁰ virus particles of AAV8 adeno-associated viruses serotype 8 (AAV8) either expressing GFP or ISM1 with a C-terminal flag tag. As predicted, the AAV8 uptake and target gene expression are highest in the liver, as confirmed by gene expression analysis of liver, BAT, skeletal muscle, iWAT tissues in these mice (FIG. 12A-D). No elevated expression of ISM1 is seen in brain (FIG. 12E). Under the same conditions, ISM1 protein levels are increased in circulation in ISM1-AAV8 mice compared with GFP-AAV8 mice, as determined by the detection of ISM1 using an antibody against the C-terminal flag tag (FIG. 5B). When these mice are fed a high fat diet, the ISM1-AAV8 mice have a blunted weight gain that is significantly different after 10 weeks of high-fat diet feeding (FIG. 5C).

Body composition analyses showed that the weight difference is entirely due to loss of fat mass and not lean mass (FIG. 12F). The weight difference could not be explained by a difference in food intake, as the accumulated food intake in the ISM1-AAV8 mice was not significantly different from the GFP mice (FIG. 12G). Whole-body energy expenditure measurements reveal no significant changes in oxygen consumption, although trending higher (FIG. 12H). These studies also indicate that ISM1-AAV8 mice have improved glucose tolerance (FIG. 5D), and dramatically increased insulin sensitivity at 10 weeks (FIG. 5E). While non-fasting glucose levels are not changed (FIG. 12I), plasma levels of insulin are lower in the ISM1-AAV8 mice, consistent with improved peripheral insulin sensitivity (FIG. 5F).

To directly assess whole-body insulin sensitivity in the ISM1-AAV8 mice, we next performed hyperinsulinemic-euglycemic clamp studies. These studies were performed at 3 weeks of high-fat diet feeding—a time point where the weight difference was not significantly different between the groups. During the clamp analysis, plasma glucose levels are adjusted between the groups to reach approximately 110 mg/ml (FIG. 5G). ISM1-AAV8 mice have an increased glucose infusion rate suggestive of increased insulin sensitivity (FIG. 5H). Interestingly, endogenous hepatic glucose production under basal and clamped conditions demonstrates a significant suppression under clamped conditions in the ISM1-AAV8 mice compared with GFP-AAV8 mice (FIG. 5I-J). Glucose levels are normally regulated by glucagon to increase the concentration of glucose and fatty acids in the bloodstream (Hilder et al., 2005); however, neither fed glucose levels nor glucagon are changed in ISM1-AAV8 mice (FIG. 12I-J). Histological analyses showed decreased lipid droplet size in BAT and iWAT from ISM1-AAV8 mice compared with GFP-AAV8 mice after 10 weeks of high fat diet feeding, consistent with a leaner phenotype (FIG. 12K). More importantly, we also observed a robust reduction in liver fat by histological analyses and Oil red O staining demonstrating reduced lipid droplet formation in ISM1-AAV8 mice (FIG. 5K). The hepatic steatosis reduction is accompanied by a strong suppression of srebp1c, and an overall trend towards suppression of usf1, fas and acc expression in ISM1-AAV8 livers, suggesting that hepatocytes could be direct targets of ISM1 (FIG. 5L). These data are further corroborated by reduced triglycerides in ISM1-AAV8 livers (FIG. 5M); however, no changes in plasma cholesterol were seen under the same conditions (FIG. 12L). In conclusion, chronic elevations of circulating ISM1 improves insulin sensitivity and hepatic steatosis in mice.

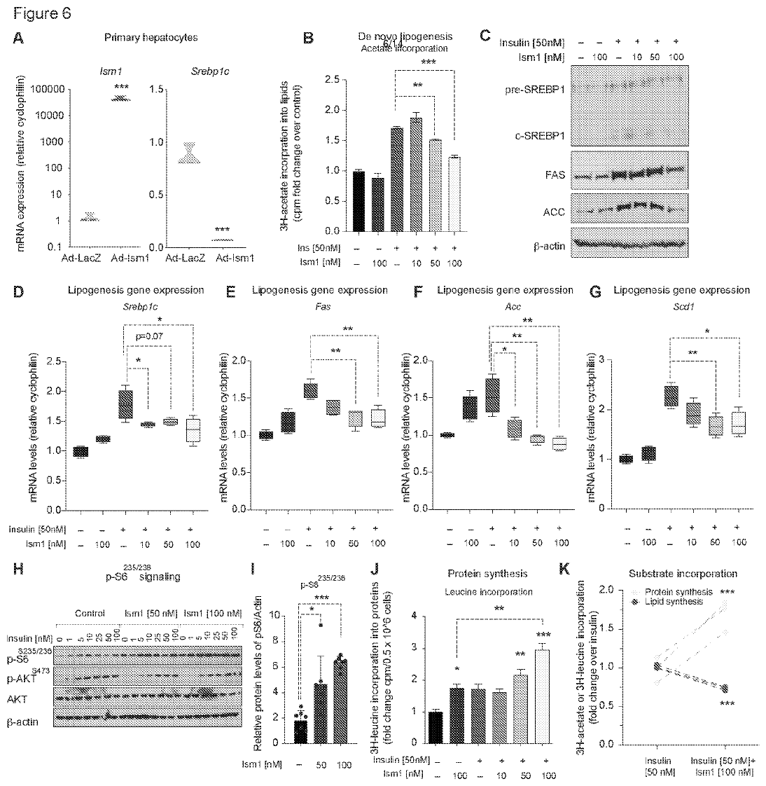

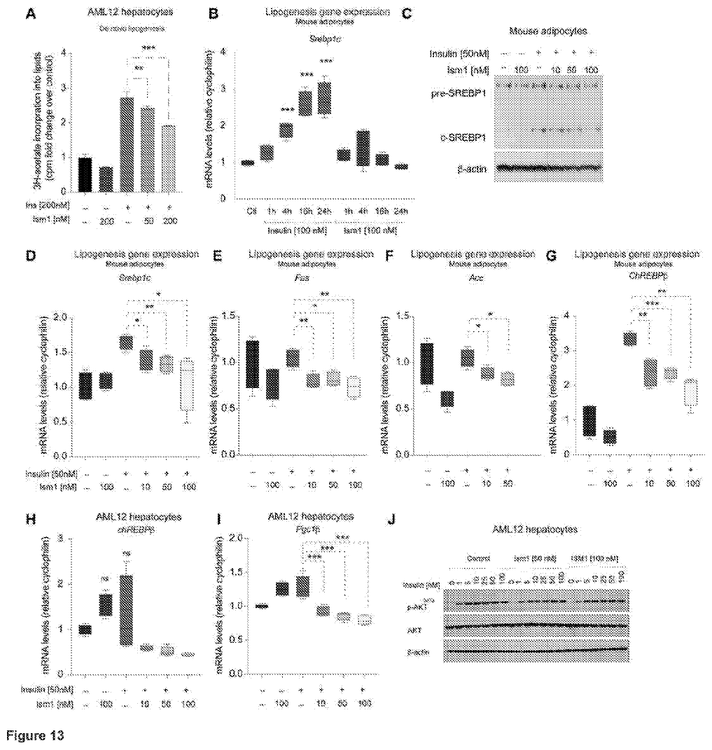

ISM1 suppresses de novo lipogenesis and increases protein synthesis in hepatocytes. Since ISM1-AAV8 overexpression largely prevents the development of hepatic steatosis, we next set out to determine whether ISM1 has a direct or indirect effect on liver lipid synthesis. To do so, we performed experiments in primary hepatocytes and the hepatocyte cell line AML12. Forced expression of ISM1 in primary hepatocytes using adenoviral vectors results in a strong suppression srebp1c transcription after 24 h, suggesting that ISM1 can directly regulate lipogenic gene expression (FIG. 6A). Insulin is well known to drive de novo lipogenesis by regulating the activity and transcription of sterol regulatory element binding protein-1c (SREBP-1c). To directly test whether ISM1's suppressive effect on srebp1c and its target genes is sufficient to attenuate hepatocyte de novo lipogenesis induced by insulin, we next performed experiments measuring H³-acetate incorporation into fatty acids and cholesterol followed by lipid extraction in the hepatocyte cell line AML12. As expected, 50 nM insulin induces a significant induction of lipogenesis, and importantly, the addition of 50 and 100 nM ISM1 could reverse the insulin-induced lipogenesis dose-dependently (FIG. 6B). Moreover, 50 nM ISM1 is sufficient to reduce lipogenesis in the presence of 200 nM supraphysiological insulin doses, demonstrating the potency of ISM1's suppressive effect (FIG. 13A). The process of de novo lipogenesis is strongly driven by insulin via the PI3K/AKT pathway leading to mTORC1 activation, which facilitates the cleavage of SREBP1c via a mechanism that still remains to be established. Therefore, we next investigated the cleavage of SREBP1c in the presence or absence of ISM1 and insulin. As expected, hepatocytes treated with 50 nM insulin show increased expression of pre-SREBP1c as well as the cleaved SREBP1, and increased protein expression of FAS and ACC (FIG. 6C). Intriguingly, we show that ISM1 counteracts the insulin-induced increase in SREBP1c cleavage to the mature form, as well as counteracts the increased target proteins FAS and ACC (FIG. 6C), suggesting a suppression of lipogenic gene and protein expression. Therefore, we directly assessed the hepatocyte gene expression of srebp1c target genes and show that fas, acc and scd1, known srebp1c target genes, are all significantly reduced by ISM1 in a dose-dependent manner in the presence of insulin (FIG. 6D-G). Similarly, in primary adipocytes, only insulin induces srebp1c expression, while we see no induction with ISM1 under basal conditions (FIG. 13B). Moreover, under insulin-stimulated conditions, ISM1 potently reduces the cleaved SREBP-1c protein levels (FIG. 13C) as well as suppressing srebp1c and the lipogenesis target genes acc, fas, and ChREBPβ (FIG. 13D-G) in adipocytes, consistent with lipogenesis suppression as a general mechanism downstream of ISM1. We cannot exclude involvement of other pathways known to control lipogenesis such as ChREBPβ and PGC1β, as these genes are also reduced upon ISM1 treatment (FIG. 13H-I).

To determine the mechanism by which ISM1 suppresses lipogenesis, we next performed acute and long-term signaling experiments in hepatocytes. Acute treatments with insulin using doses from 1 nM up to 100 nM in the presence or absence of 25 nM or 50 nM ISM1 do not result in any additional signaling changes at 5 min (FIG. 13J), but the 24 h treatment showed that ISM1 induces hyperactivation of pS6^{S235/S236}, a kinase well known to activate protein synthesis (FIG. 6H-1). This raised the question whether chronic exposure to ISM1 can switch the substrate utilization used for anabolic cellular processes, including lipid and protein synthesis. Indeed, the combined chronic ISM1 and insulin treatment that results in the sustained pS6^{S235/S236} levels leads to a 2.9-fold induction of protein synthesis as measured by H³-leucine incorporation into proteins, which is significantly higher than either of the treatments alone (FIG. 6J). Furthermore, a direct side-by-side comparison of ISM1's action in hepatocytes in the presence of insulin demonstrates that ISM1 suppresses lipid synthesis in favor of protein synthesis, strongly suggesting that ISM1 is switching the cellular anabolic state to protein synthesis (FIG. 6K). Taken together, these results demonstrate that ISM1 acts directly on hepatocytes in the presence of insulin to upregulate anabolic protein signaling pathways and protein synthesis, while suppressing srebp1c target genes and lipid synthesis.

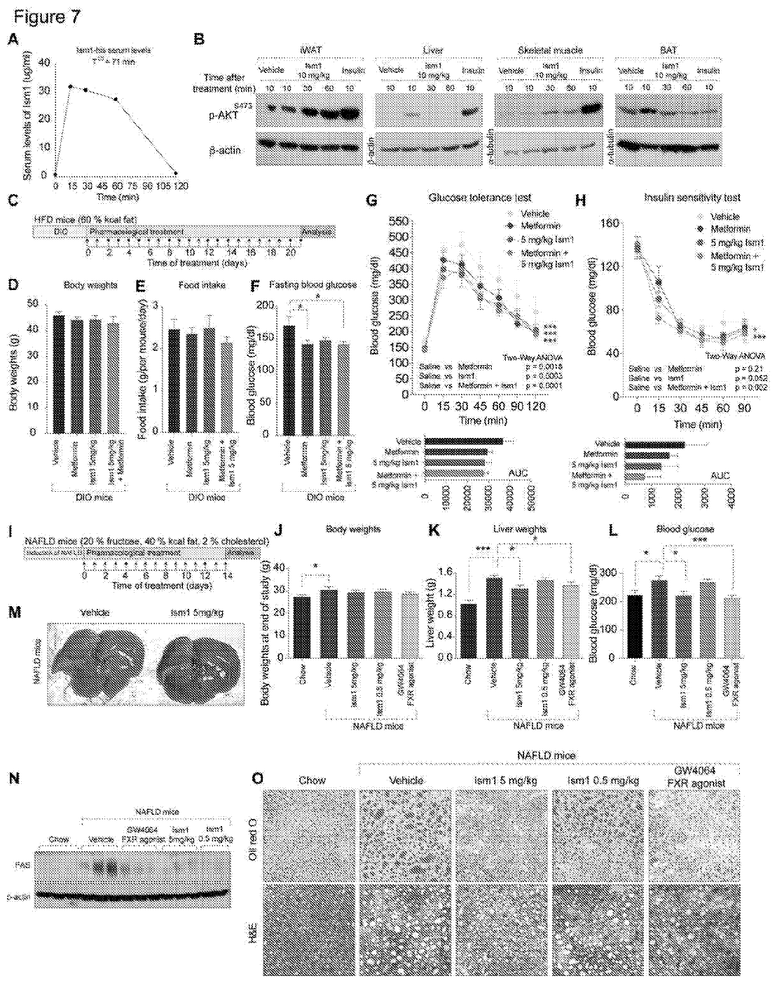

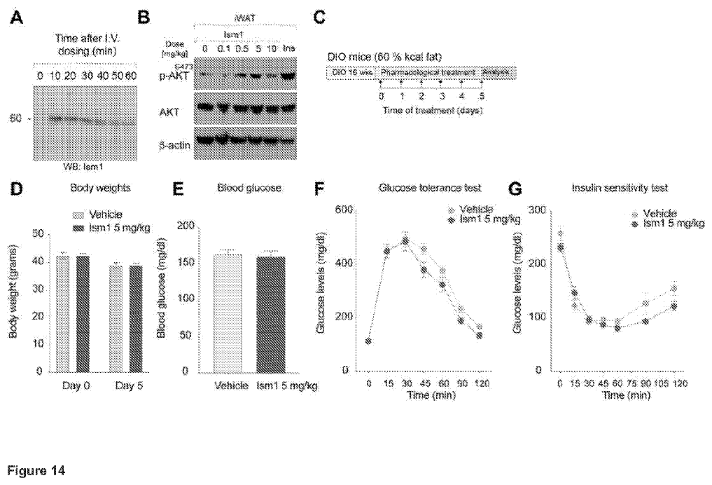

Therapeutic administration of recombinant ISM1 improves diabetes and hepatic steatosis. The above studies showing a robust signaling action of ISM1, increased adipose tissue glucose uptake, suppressed lipogenesis, and increased protein synthesis, raised the possibility of pharmacological administration of ISM1 as a therapy for diabetes and hepatic steatosis. To explore its therapeutic potential, we next determined whether therapeutic dosing of recombinant ISM1 could reverse any aspect of established metabolic disease in mice by performing a series of ISM1 administration studies benchmarked to known drugs in two different C57BL/6 mice disease models; DIO and non-alcoholic steatohepatitis (NAFLD). First, the ISM1 protein was evaluated for its pharmacokinetic properties. 10 mg/kg ISM1 protein was I.V. injected into mice and the serum levels of ISM1 were determined by an ELISA assay detecting the C-terminal his tag. This demonstrated a half-life in the blood of approximately 70 minutes (FIG. 7A) with no observed protein degradation or cleavage (FIG. 14A). To assess whether ISM1 induces AKT signaling in vivo, mice were I.V. injected with vehicle, ISM1 (10 mg/kg), or insulin (1 U/kg) and tissues were harvested at the indicated times for signaling assays. ISM1 activates pAKTS473 signaling at 30 and 60 min in iWAT and skeletal muscle, and transient inductions in BAT and liver at the 10 min time point (FIG. 7B). To determine the optimal in vivo dosing, a dose-response experiment using ISM1 doses ranging from 0.1 mg/kg to 10 mg/kg revealed that 5 mg/kg ISM1 dose induces the highest pAKTS473 response (FIG. 14B). To complement the prophylactic ISM1 overexpression experiments, we performed pilot therapeutic studies in 16-week high fat diet-fed (DIO) mice injected with vehicle or 5 mg/kg ISM1 for five days (FIG. 14C) which demonstrates no difference in body weight (FIG. 14D), ad lib glucose levels (FIG. 14E), but a modest improvement in glucose clearance (FIG. 14F) and insulin tolerance (FIG. 14G), suggestive of a therapeutic function after five days.

To evaluate the long-term therapeutic action of ISM1 to the benchmark Metformin, and also determine whether the combined treatment has an additional improvement over any of the treatments alone, 16-week DIO mice were dosed with vehicle, 5 mg/kg ISM1, 100 mg/kg Metformin, or the combined 5 mg/kg ISM1 and 100 mg/kg Metformin daily for 21 days (FIG. 7C). At the end of the experiment, there was no difference in body weights (FIG. 7D) or food intake (FIG. 7E), but fasting blood glucose was lower in all treatment groups compared with vehicle (FIG. 7F). Importantly, both ISM1, Metformin and the combined treatments have comparable effects on improving glucose tolerance compared with vehicle treated mice (FIG. 7G). Interestingly, the combined treatment performed better in the insulin sensitivity test compared with either treatment alone (FIG. 7H). These results show that therapeutic administration of recombinant ISM1 into mice improves established diabetes.

Given ISM1's suppressive effects on lipid production, we next wanted to determine whether ISM1 could reverse established non-alcoholic fatty liver disease (NAFLD) in mice. NAFLD was induced by feeding mice a 40% fat, 2% cholesterol diet for three weeks prior to treatment (FIG. 7I). Consistent with previously reported diets of similar composition, mice develop hepatic steatosis after 1-4 weeks of feeding. Mice with established NAFLD were dosed with vehicle, 0.5 mg/kg ISM1, 5 mg/kg ISM1 daily for 14 days. As a benchmark control, daily injections of the FXR agonist GW4064 at 30 mg/kg is used, which has previously been shown to significantly reduce hepatic steatosis after 14 days. Expectedly, vehicle-treated mice on NAFLD diet have higher liver weights, blood glucose and histological signs of steatosis compared with chow fed mice (FIG. 7J-N). Intriguingly, no differences are seen in body weight between the treatment groups (FIG. 7J), but mice treated with 5 mg/kg ISM1, but not 0.5 mg/kg ISM1, have reduced liver weights (FIG. 7K) and fed blood glucose compared with vehicle-treated NAFLD mice (FIG. 7L). Importantly, the effect of 5 mg/kg ISM1 was as potent as the FXR agonist GW4064 treatment in reducing liver weights, blood glucose, and histological signs of steatosis. 5 mg/kg ISM1 also shows obvious gross morphological changes in liver size and color compared with vehicle treated NAFLD mice (FIG. 7M). Furthermore, comparative analyses demonstrates that 5 mg/kg ISM1 performs equal to the FXR agonist GW4064 in reversing hepatic steatosis, as determined by reduced hepatic Fatty Acid Synthase (FAS) protein levels (FIG. 7N), as well as reduced Oil red O staining (FIG. 7O). In conclusion, these data show that pharmacological administration of ISM1 improves glucose tolerance and reverses established hepatic steatosis in mice.

Taken together, using several orthogonal genetic and pharmacologic methods, we have identified a bioactive secreted hormone with overlapping, but distinct signaling activities with insulin. Importantly, because ISM1 counteracts de novo lipogenesis and switches hepatocytes from a lipogenic to a protein synthesis state, ISM1 is therapeutic for fatty liver disease.

A range of drugs are currently available for type 2 diabetes, but there is an unmet need for drugs targeting diabetes and non-alcoholic fatty liver disease simultaneously. Here, we demonstrate that ISM1 is a secreted polypeptide hormone that regulates adipose tissue glucose uptake while reducing steatosis in the liver. Thus, pharmacologically targeting the ISM1 pathway provides increased glucose uptake without causing the often-accompanied side effects of hepatic steatosis and weight gain seen with insulin or insulin-sensitizing therapies. The mechanism of ISM1 action is unusual and intriguing. Surprisingly, while pAKT^{S473} activation by insulin activates lipogenesis to promote fat storage in adipose or liver tissues, ISM1 reduces de novo lipogenesis and increases protein synthesis, thus dissociating the canonical pathways induced by insulin. The increased protein synthesis suggests that ISM1, in the presence of insulin compared with insulin alone, enables a cellular metabolic switch mediated by pS6^{S235/S236}. Importantly, this slight divergence in intracellular signaling pathways apparently has major functional consequences. The increase in hepatocyte protein synthesis by ISM1 is reminiscent of the hepatic actions of FGF19, which stimulates protein synthesis and glycogen synthesis while inhibiting lipid synthesis in the liver.

Some insulin-independent glucoregulatory processes can cause unwanted hypoglycemia, while others, such as FGF1, do not appear to lead to hypoglycemia. In our studies with overexpression or pharmacological administration of ISM1, we do not observe hypoglycemia, showing that counter regulatory mechanisms exist. Administration of ISM1 into mice with established disease ameliorates glucose and lipid dysfunction. In conclusion, the uncovered ISM1 action represents an unexpected ligand-induced signaling pathway with metabolic effects on multiple organ systems. Given ISM1's dual beneficial effects on glucose homeostasis and lipid-lowering properties, recombinant ISM1 and its derivatives have therapeutic purposes.

### Experimental Model and Subject Details

Mouse models. Animal experiments were performed per procedures approved by the Institutional Animal Care and Use Committee of the Stanford Animal Care and Use Committee (APLAC) protocol number #32982. Experiments were performed according to procedures approved by the Institutional Animal Care and Use Committee of the Beth Israel Deaconess Medical Center and Yale University School of Medicine IACUC. C57BL/6J male and female mice were purchased from the Jackson Laboratory (#000664). Unless otherwise stated, mice were housed in a temperature-controlled (20-22° C.) room on a 12-hour light/dark cycle. All experiments were performed with sex- and age-matched male mice housed in groups of five unless stated otherwise. This study generated a new ISM1-KO mouse model. The Ism1 floxed allele targeting intron 1 and intron 4 of the Ism1 gene to delete exon 2-4 was generated using CRISPR-mediated gene editing (Applied Stem Cell). The ISM1 flox mice were crossed with female mice expressing the Ella-Cre transgene for embryonic deletion. The deletion of the exons was confirmed with pcr for the exon junction, Sanger sequencing, and q-pcr. The following genotyping primers were used: Ism1 KO: 5′-CTATGCTATGCCCAGTGTCTCTCTCTG-3′; 5′-CAAACTGACCAGAGTCCCTCCTTCAA-3′, Ism11 WT: 5′-GAACACTGAGGAAGTTGCTGTCA-3′; 5′-ATGGCCCTGACTCCGAAGCAGAA-3. Total RNA was extracted from adult mice and reverse-transcription was performed as described above. qPCR was performed using the following primers: Ism11-F: 5′-FAGAGCAGCCAGAGTATGATTCC-3′ and Ism11-R: 5′-RGCCGCTGTCCTGAAAGTATCT-3.

Human samples. For transcriptional analyses on human adipose samples, subcutaneous adipose tissue was collected under IRB 2011 P000079 (approved by the Beth Israel Deaconess Medical Center Committee on Clinical Investigations) from subjects recruited from the plastic-surgeon operating-room schedule at Beth Israel Deaconess Medical Center in a consecutive fashion, as scheduling permitted, to process the sample. The inclusion criteria were healthy male and female subjects, ages 18-64 receiving abdominal surgery. The exclusion criteria were diagnosis of diabetes, any subjects taking insulin-sensitizing medications such as thiazolidinediones or metformin, chromatin-modifying enzymes such as valproic acid, and drugs known to induce insulin resistance such as mTOR inhibitors (for example, sirolimus or tacrolimus) or systemic steroid medications. Fasting serum was collected and tested for insulin, glucose, free fatty acids, and a lipid-panel was performed in a Clinical Laboratory Improvement Amendments approved laboratory. BMI measures were derived from electronic medical records and confirmed by self-reporting, and measures of insulin resistance, the homeostasis model assessment-estimated insulin resistance index (HOMA-IR) and revised quantitative insulin sensitivity check index (QUICKI) were calculated. Female subjects in the first and fourth quartiles for either HOMA-IR or QUICKI and matched for age and BMI were processed for RNA-seq. Human participants who donated adipose tissue provided informed consent. Human plasma was obtained from 11 female individuals from the single-site, randomized crossover SWAP-MEAT Trial (NCT03718988) (Crimarco et al., 2020). Glucose levels were only available for 8 out of 11 samples. The inclusion criteria were healthy individuals over 18 years of age. Exclusion criteria were weighing <110 lbs, BMI >40, LDL cholesterol >190 mg/dl, systolic blood pressure >160 mm Hg or diastolic blood pressure >90 mm Hg, as well as other clinically significant diseases. All samples were blinded and analyzed by ISM1 ELISA.

Cell lines and reagents. AML12 mouse hepatocytes were purchased from ATCC (#CRL-2254) and cultured with DMEM/F12 medium (Gibco) supplemented with 10% FBS, 10 μg/ml insulin, 5.5 μg/ml transferrin, 5 ng/ml selenium, 40 ng/ml dexamethasone, and 15 mM HEPES. 3T3-F332A cells (Sigma, Cat #00070654), 3T3-L1 cells (ATCC, Cat #CL-173), primary human skeletal muscle cells (Cook Myocytes, Cat #SK-1111), Expi293F cells (ThermoFisher #Cat #A14527) were cultured according to manufacturer's instructions. SGBS cells were obtained from Wabitsch laboratory and cultured as previously described (Fischer-Posovszky et al., 2008; Wabitsch et al., 2001). All cells were cultured in a humidified atmosphere containing 5% CO2 at 37° C.

Mouse and human pre-adipocytes culture and differentiation. Inguinal fat pads from 4-8 week old C57BL/6J male and female mice were dissected and mechanically digested for 10 min using spring scissors. Digested tissues were incubated in WAT isolation buffer (10 ml PBS, 2.4 U/ml dispase II (#04942078001, Roche), 10 mg/ml collagenase D (#11088858001, Roche) at 37° C. for 45 min. 20 mL growth media was added and the tissue suspension was filtered through a 100-μm cell strainer and centrifuged at 600×g for 5 min. The cell pellets were resuspended in 20 mL growth media, filtered through a 40-μm cell strainer, centrifuged at 600×g for 5 min, resuspended in 10 mL growth media, and plated in 10-cm collagen-coated dishes. The cells were cultured in growth media (DMEM/F-12 Glutamax, Thermo Fisher Scientific #10565018) supplemented with 10% fetal bovine serum. Two days post-confluency, differentiation was induced with growth media containing 1 μM rosiglitazone, 0.5 mM isobutylmethylxanthine, 1 μM dexamethasone, 5 μg/mL insulin. After two days, cells were re-fed with growth media containing 1 μM rosiglitazone and 5 μg/mL insulin. Cells were fully differentiated after 6-8 days. For ISM1 knockdown or overexpression in primary adipocytes using adenovirus, 500 μL crude virus was mixed with 500 μL fresh growth media containing 1 μM rosiglitazone and 5 μg/mL insulin was added to each well at day 2 of differentiation. At day 4, virus-containing media was removed and 500 μL fresh growth media without stimulators was added to each well. SGBS cells were differentiated as described previously (Fischer-Posovszky et al., 2008; Wabitsch et al., 2001). Briefly, cells were cultured in growth media DMEM/F-12 Glutamax, supplemented with 10% fetal bovine serum, 33 uM biotin, and 17 uM pantothenate. Two days post confluency, differentiation was induced with serum-free DMEM/F12 media containing 33 uM biotin, 17 uM pantothenate, 0.01 mg/ml transferrin, 20 nM insulin, 100 nM cortisol, 0.2 nM triiodothyronine, 25 nM dexamethasone, 0.25 mM isobutyl methylxanthine, and 2 μM rosiglitazone. After four days, cells were re-fed with growth media containing 0.01 mg/ml transferrin 20 nM insulin, 100 nM cortisol, 0.2 nM triiodothyronine. Cells were fully differentiated after day 8-10.

Primary mouse hepatocyte isolation and culture. Primary hepatocyte isolation was performed as previously described. Briefly, 4-8 week old C57BL/6J mice were sacrificed, and livers were perfused in HBSS buffer (#14175-095, Gibco) supplemented with 0.4 g/L KCl, 1 g/L glucose, 2.1 g/L sodium bicarbonate, and 0.2 g/L EDTA for 3 minutes, followed by Collagenase (#C5138, Sigma) digestion at 37° C. Cells were dissociated from the digested livers and hepatocytes were suspended in Williams Medium E (#112-033-101, Quality Biological) supplemented with 10% FBS, 2 mM sodium pyruvate, 1 μM dexamethasone, and 100 nM insulin (plating medium). The cell suspension was filtered through a 70 μm strainer and centrifuged at 50×g for 3 minutes. Cell pellets were resuspended in plating medium and mixed with 90% Percoll (#P1644, Sigma) followed by centrifugation at 100×g for 10 minutes. Cell pellets were washed and resuspended in plating medium. 4 hours after seeding on collagen-coated plates, hepatocytes were washed with PBS, followed by the addition of Williams E supplemented with 0.2% BSA, 2 mM sodium pyruvate, 0.1 μM dexamethasone (maintenance medium).

### Method Details

ISM1 overexpression in vivo using AAV8. Adeno-associated virus serotype 8 expressing mouse ISM1 with a C-terminal flag tag (AAV8-Ism1-flag) was made by Vector Biolabs and the AAV8-GFP (#7061) control was purchased at the same time. 8-week-old mice were subjected to I.V. injection of 10¹⁰ virus particles/mouse of AAV8-Ism1-flag or AAV8-GFP diluted in saline in a total volume of 100 μL. After injection, mice were fed a high-fat diet (60% fat, Research Diets). Body weights were measured and recorded every week. After 10 weeks of high-fat diet feeding, mice were subjected to glucose tolerance tests and insulin tolerance tests. Tissues were collected for gene expression and histological studies at the weeks indicated. Plasma was collected for detecting plasma levels of ISM1-flag, glucagon, and insulin.

Pharmacokinetic measurements of ISM1 blood levels following ISM1 administration. An anti-his tag ELISA was performed to measure ISM1-his concentrations in blood. Recombinant ISM1 protein was administered at 10 mg/kg I.V. in a total volume of 100 ul. Blood was collected and centrifuged at 6000×g for 10 minutes at room temperature to collect serum. Blood levels were measured using the HisProbe-HRP Conjugate according to the manufacturer's instructions (Thermo #15165). Briefly, samples were prepared by diluting serum in coating buffer at 1:100 ratio and added to wells for overnight incubation at 4° C. Nonspecific binding was blocked by adding blocking buffer and incubating for 30 min at 37° C. The plate was washed three times with wash buffer. HisProbe-HRP (Thermo, #15165) working solution was added to each well. After 15 min incubation at room temperature, the plate was washed four times with wash buffer, followed by adding substrate (Thermo, #34022). After 10 min, 1N sulfuric acid was added to stop the reaction. Absorbance was measured at 450 nm.

Therapeutic ISM1 recombinant protein administration in vivo. All pharmacologic studies using recombinant ISM1 protein were performed in mice with established diet-induced obesity or non-alcoholic fatty liver disease (NAFLD). For all experiments, male C57BL/6J mice purchased from Jax were fed either a high-fat diet (#D12492, Research Diets) or a NAFLD diet (#D09100310, Research Diets) prior to ISM1 protein injections. In all experiments, mice were mock injected with saline for three days prior to protein or drug injections to prevent stress-induced weight loss. Mice were I.P. injected with vehicle (saline) or indicated doses of ISM1 protein diluted in saline. For the 14-day experiments in NAFLD mice, 7-weeks old mice were fed with NAFLD diet for 3 weeks and then mock injected with saline for 3 days prior to protein injections. The induction of lipid accumulation with NAFLD diet was verified by the significant increase in hepatic lipid levels compared with mice on chow diet at the same time point. NAFLD mice were then daily I.P. injected with either vehicle (saline containing 5% DMSO and 10% Kolliphor) or indicated doses of ISM1 protein (500 μg/kg or 5 mg/kg) or with FXR agonist (30 mg/kg, GW4064, Sigma-Aldrich, #G5172) diluted in vehicle (saline containing 5% DMSO and 10% Kolliphor). For the 21-day experiments in HFD mice, mice were mock injected with saline for three days prior to protein injections. Mice were then daily I.P. injected with either vehicle (saline) or 5 mg/kg of ISM1 protein diluted in saline. Metformin (100 mg/kg, Sigma, #317240) was diluted in saline and daily administered by oral gavage. All groups received control injections by I.P. and oral gavage. At the end of the experiments, mice and tissue weights were recorded. Tissues and plasma were collected and frozen for further analyses.

In vivo glucose uptake. Mice were fasted for 1 h and injected I.P. with 3H-2-deoxyglucose (3H-2-DOG) at 100 uCi/kg with or without 0.75 U/kg insulin in a total volume of 120 ul per mouse. After 30 min, mice were euthanized. Blood was collected by cardiac puncture and subsequently centrifuged to collect serum. Wet tissue weights were recorded then homogenized in 1% SDS for liquid scintillation counting. Data are expressed as CPM/mg wet weight (fold change over control).

Glucose tolerance and insulin tolerance tests. For glucose tolerance tests, mice were fasted overnight and I.P. injected with glucose at 2 g/kg body weight. Blood glucose levels were measured at 0, 15, 30, 45, 60, 90 and 120 mins. For insulin tolerance tests, mice were fasted for 2 h, and I.P. injected with 0.75 U/kg insulin. Blood glucose levels were measured at 0, 15, 30, 45, 60, 90 and 120 mins.

Body temperature measurements. Adult mice were implanted with a subcutaneous temperature probe (IPTT Temperature Transponder, Bio Medic Data Systems, Inc.). Mice were group-housed at room temperature for the entire duration of the experiments. Temperature probes were allowed to stabilize for 3 days until the first temperature was measured. During measurements, the body temperature was recorded using an IPTT scanner in conscious mice in their home cages.

Food intake, energy expenditure and body composition measurements. Measurements of accumulated food intake and VO2 were performed using a Comprehensive Lab Animal Monitoring System at room temperature (20-22° C.) (Oxymax, Columbus Instruments) as previously described (Cohen et al., 2014; Long et al., 2016; Svensson et al., 2016). Singly housed mice were acclimated in metabolic chambers for at least 24 h before the start of experiments to minimize stress. Fat mass, lean mass, and water mass were measured by Echo MRI.

Construction of shRNA-pENTR/U6 Entry Vector of ISM1. To generate small hairpin RNA (shRNA)-expressing pENTR/U6 entry constructs, single-stranded DNA Oligo sequences encoding the shRNA of mouse Ism1 were designed following the guidelines of the BLOCK-it U6 RNAi Entry Vector kit (#K494500, Thermo Fisher Scientific). Two sets of complementary oligonucleotide strands were designed based on the following sequences:

top strand: 5′-CACCGGTCACCATAGAGGTGGTTGACGAATCAACCACCTCTATGGTGACC-3′, bottom strand: 5′-AAAAGGTCACCATAGAGGTGGTTGATTCGTCAACCACCTCTATGGTGACC-3′, and top strand: 5′-CACCGCGGGAAGTGAGGAGTTTAATCGAAATTAAACTCCTCACTTCCCGC-3′, bottom strand: 5′-AAAAGCGGGAAGTGAGGAGTTTAATTTCGATTAAACTCCTCACTTCCCGC-3′. Non-targeting lacZ control dsDNA oligos were included in the kit. The single-stranded DNA oligos were annealed and cloned into pENTR/U6 vectors included in the kit following the manufacturer's instructions. Briefly, 1 nmol of each complementary oligonucleotide strand and 2 μL of 10× denaturation buffer solution was mixed and sterilized deionized H2O was added to a final volume of 20 μL. The reaction mixture was denatured at 95° C. for 4 min, then annealed at room temperature for 10 min. Ds-oligos were diluted with 1× oligo annealing buffer at a final concentration of 5 nM. T4 DNA ligase was used to clone the ds-oligos into the pENTR/U6 vector. 2 μL of ligation reaction was transformed into One Shot® TOP10 chemically competent E. coli cells. Selected clones were sequenced by using primers provided with the kit.

Generation of shRNA-expressing Adenoviral Destination Clones. The BLOCK-iT Adenoviral RNAi Expression System (#K494100, Thermo Fisher Scientific) was used to generate adenoviral shRNA destination clones according to the manufacturer's instructions. Briefly, pENTR/U6 entry clones were transferred into adenoviral pAd/BLOCK-iT™-DEST destination vectors by performing LR recombination reactions with Gateway LR Clonase II followed by transformation into One Shot® TOP10 chemically competent E. coli cells. The constructs for recombinant shRNA-expressing adenoviral destination clones were purified using the Qiagen Plasmid Midi kit (catalog number 12143, Qiagen). Selected clones were sequenced by using the following primers: F: 5′-GACTTTGACCGTTTACGTGGAGAC-3′ and R: 5′-CCTTAAGCCACGCCCACACATTTC-3′.

Adenovirus Production. To produce crude shRNA-adenoviral stocks, 15 μg of plasmid was digested with PacI-restriction enzyme and purified with QiAquick PCR purification kit (#28104, Qiagen). HEK 293A cells were seeded at 5×10⁵ cells/well in a 6-well plate the day before transfection. Cells were transfected with 3 μg of PacI-digested shRNA adenovirus plasmids and LipoD293 transfection reagent (SignaGen Laboratories). The media was replaced ˜16 hours post-transfection. 48 hours post-transfection, cells were trypsinized and transferred into a 10 cm dish. Adenovirus-containing cells and medium were harvested and transferred into a 15 ml Falcon tube after 7-9 days post-transfection when cytopathic effects were observed in more than 80% of the cells. The crude adenovirus stocks were prepared by three freeze-thaws, followed by centrifugation to remove cell debris. Crude adenoviral stocks were stored at −80° C. in 1 ml aliquots. The crude virus stocks were further amplified by infection of HEK 293A cells grown in three 15-cm dishes (300 μL of crude virus/15 cm dish). Three days later, adenovirus-containing cells and medium were harvested and centrifuged as described for crude virus. Amplified viral stocks were stored in aliquots at −80° C.

Expression, purification, and characterization of recombinant ISM1 protein. The proteins used in this study were generated by transient transfection of a mouse ISM1-flag or ISM1-myc-his DNA plasmid in mammalian Expi93F cells. The protein was purified using a Ni column and buffer exchanged to PBS. Protein purity and integrity were assessed with SDS page, Superdex200 size exclusion column, endotoxin assays, western blot, and mass spectrometry. Every protein batch was tested for bioactivity using pAKT^{S473} as the readout. Following purification, the protein was aliquoted and stored at −80° C. and not used for more than three freeze-thaws. For protein deglycosylation, the reaction was performed according to the manufacturer's protocol using deglycosylation Mix II from New England BioLabs (#P6044). Briefly, 25 μg of ISM1 protein or control protein fetuin was dissolved in 40 μL water. 5 μL Deglycosylation Mix Buffer 2 was added to the protein solution, and the protein solution was incubated at 75° C. for 10 minutes. 5 μL Protein Deglycosylation Mix II was added to the protein solution followed by a 30-minute incubation at room temperature and 1 h at 37° C. The deglycosylated proteins were analyzed by silver stain and western blot.

Measurements of ISM1 protein stability using transmittance. Briefly, ISM1 or insulin was plated at 150 μL per well (n=3/group) in a clear 96-well plate and sealed with optically clear and thermally stable seal (VWR). The plate was immediately placed into a plate reader and incubated with continuous shaking at 37° C. Absorbance readings were taken every 10 minutes at 540 nm for 100 h (BioTek SynergyH1 microplate reader). Values are expressed as total transmittance.

SureFire Ultra AKT1/2/3 (pS473) AlphaLISA. AKT 1/2/3 (pS473) AlplaLISA kit was purchased from PerkinElmer (#ALSU-PAKT-B-HV) and serine 473 phosphorylation of AKT was measured according to the instructions in the protocol provided by the manufacturer. Briefly, confluent F442A cells were starved overnight in serum-free DMEM/F12 media. On the following day, insulin or ISM1 were added to cells. After a 5 min incubation, cells were washed with cold PBS once and lysed using lysis buffer supplied in the kit. 30 μL of cell lysate was transferred, followed by incubation with 15 μL of Acceptor Mix for 1 hr at room temperature. 15 μL of Donor Mix was added to each well and the plate was incubated at room temperature for 1 hr in the dark. The Alpha signals were measured on Tecan Infinite M1000 Pro plate reader using standard AlphaLISA settings.

Gene expression analysis. Total RNA from cultured cells or tissues was isolated using TRIzol (Thermo Fischer Scientific) and Rneasy mini kits (QIAGEN). RNA was reverse transcribed using the ABI high capacity cDNA synthesis kit. For qRT-PCR analysis, cDNA, primers and SYBR-green fluorescent dye (ABI) were used. Relative mRNA expression was determined by normalization with Cyclophilin levels using the ΔΔCt method.

Western blots and molecular analyses. For western blotting, homogenized tissues or whole-cell lysates, samples were lysed in RIPA buffer containing protease inhibitor cocktail (Roche) and phosphatase inhibitor cocktail (Roche). Cell lysates were centrifuged at 14,000×g for 15 min and supernatants were prepared in 4×LDS Sample Buffer (Invitrogen) and separated by SDS-PAGE and transferred to Immobilon 0.45 μm membranes (Millipore). For Western blotting of plasma samples, 1 μl of plasma was prepared in 2× sample buffer (Invitrogen) with reducing agent, boiled, and analyzed using Western blot against indicated antibody. Protein kinase array (R&D systems ARY003B) was performed according to the manufacturers' description. All antibodies used in this paper are described in STAR methods.

Biochemical analyses. Plasma insulin levels were measured using the Ultra-Sensitive Mouse Insulin ELISA kit (Crystal Chem Inc #90080). Plasma triglycerides were measured with Infinity triglyceride measurement kit (Thermo #TR22421) and tissue triglycerides were measured with a Triglyceride Colorimetric Assay Kit (Cayman #10010303). Lipolysis was measured in day 5 differentiated adipocytes after 4 hours of protein treatment using a glycerol release assay according to the protocol provided by the manufacturer (Abcam #ab133130). Plasma membrane protein extractions were performed according to the manufacturer's protocol (Abcam #ab65400).

Immunocytochemistry. Primary mouse preadipocytes were seeded onto collagen-coated 22 mm×22 mm coverslips and differentiated using the method above. On day 8, differentiated adipocytes were washed with PBS and starved in 0.5% BSA in KRH buffer (50 mM HEPES, 136 mM NaCl, 1.25 mM MgSO₄, 1.25 mM CaCl₂, 4.7 mM KCl, pH 7.4) for 3.5 h. Cells were treated with insulin or ISM1 for 30 min, followed by three washes with cold PBS. Cells were fixed with 4% PFA in PBS for 15 min at room temperature. After blocking with 2% BSA, 5% goat serum, 0.2% Triton-X in PBS for 1 hr, cells were incubated with diluted primary antibody at 4° C. overnight. The next day, cells were washed and incubated with secondary antibody on ice for 1 hr. Cells were imaged using confocal microscope (Leica TCS SP8). Hoechst (#33342, thermo fisher) was used as counterstain.

Immunohistochemistry. For H&E staining, livers were formalin-fixed, paraffin-embedded, and sectioned at 6 μm. For hematoxylin and eosin (H&E) staining, sections were deparaffinized and dehydrated with xylenes and ethanol. Briefly, slides were stained with hematoxylin, washed with water and 95% ethanol, and stained with eosin for 30 min. Sections were then incubated with ethanol and xylene, and mounted with mounting medium. For Oil red O stainings, frozen liver tissue slides were fixed with 3% formalin in PBS, washed twice with water, incubated with 60% isopropanol for 5 min and then incubated with Oil Red O solution (3:2 ratio of Oil Red O:H2O) for 20 min. Immunohistochemical stainings were observed with a Nikon 80i upright light microscope using a 40× objective lens. Digital images were captured with a Nikon Digital Sight DS-Fi1 color camera and NIS-Elements acquisition software.

Glucose uptake. Glucose uptake was performed as previously described (You et al., 2017). Briefly, 2-Deoxy-d-[2,6-3H]-glucose was purchased from PerkinElmer NEN radiochemicals. Fully differentiated adipocytes were washed with KRH buffer and starved in 0.5% BSA in KRH buffer for 4 h. Indicated concentrations of insulin or ISM1 were added at indicated time points. Cells were treated with a mixture of 500 μM 2-deoxy-D-glucose and 1.71 μCimL⁻¹ 2-Deoxy-D-[1,2-3H (N)]-glucose for another 10 min followed by three washes using ice cold KRH buffer containing 200 mM glucose. The passive glucose uptake in the presence of 50 uM CytoB was less than 12% of the total glucose uptake. Cells were solubilized in 1% SDS and radioactivity was measured by liquid scintillation counting.

Lipogenesis Assay. Primary hepatocytes or AML12 hepatocytes were washed twice with warm PBS and starved in serum-free DMEM overnight. Indicated concentrations of insulin with or without ISM1 were added at the same time. After 24 h, a mixture of 10 μM cold acetate and 2 μCi [3H]-Acetate (#NET003H005MC, PerkinElmer) was added to each well and cells were incubated for another 4 hr. Cells were washed with PBS twice and lysed using 0.1 N hydrogen chloride. Lipids were extracted by 2:1 chloroform-methanol (v/v). After centrifugation at 3000×g for 10 min, the lower phase was transferred to scintillation vials and radioactivity was measured by liquid scintillation counting.

Protein synthesis Assay. AML12 hepatocytes were washed twice with warm PBS and starved in serum-free media overnight. Indicated concentrations of insulin with or without ISM1 and 0.25 uCi of [3H]-Leucine (#NET460250UC, PerkinElmer) were added at the same time. After a 24 h incubation, cells were washed three times with ice cold PBS and lysed in RIPA buffer containing protease inhibitor cocktail (Roche). After 15 min centrifugation at 14000×g, supernatants were transferred to new tubes. Trichloroacetic acid was added to the protein extracts at a final concentration of 10% and incubated on ice for 1 h. After a 15 min centrifugation at 14000×g, pellets were washed with cold acetone, diluted in 10M NaOH and transferred to scintillation vials. The radioactivity was measured by liquid scintillation counting.

Cellular respiration assay. Cells were seeded in seahorse cell culture microplates (Agilent, #100777-004) using growth media. XFe24 extracellular flux assay kit cartridge (Agilent, #102340-100) was hydrated with XF calibrant (Agilent, #100840-000) and PBS and incubated overnight in C02-free incubator. The next day, cells were washed with seahorse assay buffer (0.3M NaCl, 1 mM pyruvate and 20 mM glucose, DMEM (Sigma, D5030-10L), pen-strep, pH 7.4) and incubated in seahorse assay buffer for 1 hr in C02-free incubator. PBS or ISM1 proteins were injected into the port as indicated in the figure. The seahorse program was run with 3 cycles with a 4 min mix, 0 min wait, and a 2 min measure between injections of compounds.

LC-MS/MS. Samples were run on a 4-12% SDS-PAGE gel and resolved by Coomassie staining. Gel bands were excised and placed in 1.5 ml Eppendorf tubes and then cut in 1×1 mm squares. The excised gel pieces were then reduced with 5 mM DTT, 50 mM ammonium bicarbonate at 55° C. for 30 min. Residual solvent was removed and alkylation was performed using 10 mM acrylamide in 50 mM ammonium bicarbonate for 30 min at room temperature. The gel pieces were rinsed 2 times with 50% acetonitrile, 50 mM ammonium bicarbonate and placed in a speed vac for 5 min. Digestion was performed with Trypsin/LysC (Promega) in the presence of 0.02% protease max (Promega) in both a standard overnight digest at 37° C. Samples were centrifuged and the solvent including peptides was collected and further peptide extraction was performed by the addition of 60% acetonitrile, 39.9% water, 0.1% formic acid and incubation for 10-15 min. The peptide pools were dried in a speed vac. Samples were reconstituted in 12 μl reconstitution buffer (2% acetonitrile with 0.1% Formic acid) and 3 μl (100 ng) of it was injected on the instrument.

Mass spectrometry experiments were performed using a Q Exactive HF-X Hybrid Quadrupole-Orbitrap mass spectrometer (Thermo Scientific, San Jose, CA) with liquid chromatography using a Nanoacquity UPLC (Waters Corporation, Milford, MA). For a typical LCMS experiment, a flow rate of 600 nL/min was used, where mobile phase A was 0.2% formic acid in water and mobile phase B was 0.2% formic acid in acetonitrile. Analytical columns were prepared in-house with an I.D. of 100 microns packed with Magic 1.8 micron 120A UChrom C18 stationary phase (nanoLCMS Solutions) to a length of ˜25 cm. Peptides were directly injected onto the analytical column using a gradient (2-45% B, followed by a high-B wash) of 80 min. The mass spectrometer was operated in a data dependent fashion using HCD fragmentation for MS/MS spectra generation. For data analysis, the .RAW data files were processed using Byonic v3.2.0 (Protein Metrics, San Carlos, CA) to identify peptides and infer proteins using Mus musculus database from Uniprot. Proteolysis was assumed to be semi-specific allowing for N-ragged cleavage with up to two missed cleavage sites. Precursor and fragment mass accuracies were held within 12 ppm. Proteins were held to a false discovery rate of 1%, using standard approaches.

Human adipose tissue transcriptomics analyses. For purification of mature human adipocytes for RNA sequencing, whole-tissue subcutaneous adipose specimens were freshly collected from the operating room. Skin was removed, and adipose tissue was cut into 1- to 2-inch pieces and rinsed thoroughly with 37° C. PBS to remove blood. Cleaned adipose tissue pieces were quickly minced with an electric grinder with 3/16-inch hole plate, and 400 ml of sample was placed in a 2-1 wide-mouthed Erlenmeyer culture flask with 100 ml of freshly prepared blendzyme (Roche Liberase™, research grade, cat. no. 05401127001, in PBS, at a ratio of 6.25 mg per 50 ml) and shaken in a 37° C. shaking incubator at 120 r.p.m. for 15-20 min to digest until the sample appeared uniform. Digestion was stopped with 100 ml of freshly made KRB (5.5 mM glucose, 137 mM NaCl, 15 mM HEPES, 5 mM KCl, 1.25 mM CaCl2, 0.44 mM KH2PO4, 0.34 mM Na2HPO4 and 0.8 mM MgSO4), supplemented with 2% BSA. Digested tissue was filtered through a 300-μM sieve and washed with KRB/albumin and flow through until only connective tissue remained. Samples were centrifuged at 233 g for 5 min at room temperature, clear lipid was later removed, and floated adipocyte supernatant was collected, divided into aliquots and flash-frozen in liquid nitrogen. RNA isolation from mature human adipocytes. Total RNA from ˜400 μl of thawed floated adipocytes was isolated in TRIzol reagent (Invitrogen) according to the manufacturer's instructions. For RNA-seq library construction, mRNA was purified from 100 ng of total RNA by using a Ribo-Zero rRNA removal kit (Epicentre) to deplete ribosomal RNA and convert into double-stranded complementary DNA by using an NEBNext mRNA Second Strand Synthesis Module (E6111 L). cDNA was subsequently tagmented and amplified for 12 cycles by using a Nextera XT DNA Library Preparation Kit (Illumina FC-131). Sequencing libraries were analysed with Qubit and Agilent Bioanalyzer, pooled at a final loading concentration of 1.8 μM and sequenced on a NextSeq500. Sequencing reads were demultiplexed by using bcl2fastq and aligned to the mm10 mouse genome by using HISAT2. PCR duplicates and low-quality reads were removed by Picard. Filtered reads were assigned to the annotated transcriptome and quantified by using featureCounts.

ISM1 sandwich ELISA. ISM1 sandwich ELISA was performed using mouse serum from ISM1-KO and WT mice and on human plasma from female individuals from the single-site, randomized crossover SWAP-MEAT Trial (NCT03718988) (Crimarco et al., 2020). Anti-ISM1 antibodies were custom generated by Genscript using the recombinant mouse ISM1 protein as antigen. Antibody production was carried out for five clones separately. The antibodies were purified by mouse ISM1 A/G affinity column chromatography and dialyzed into PBS for storage. Biotin conjugation was performed on purified antibodies from each clone. For ISM1 ELISA on human or mouse plasma samples, the anti-ISM1 capture antibody was diluted (1:500) in PBS and the ELISA plate was coated with 100 μl of the antibody dilution at 4° C. for 48 h. After one wash with 260 μl PBS-T, 150 ul of blocking buffer (1% BSA in PBS) was added to each coated well and the plate was incubated at 37° C. for 2 h, dried, and incubated again at 37° C. overnight. After four washes, 200 μl of antigen diluted in sample buffer was added to each well and the plate was incubated at 4° C. overnight. After four washes, 100 μl of the diluted (1:1000) anti-ISM1 biotinylated detection antibody was added to each well and the plate was incubated at 37° C. for 2 h and then at 4° C. overnight. After four washes, Streptavidin-HRP was diluted with sample buffer to 0.2 μl/mL and 100 ul was added to the plate. The plate was incubated at 37° C. for 10 min. After five washes, 100 μl TMB reagent was added to each well and the plate was incubated at room temperature for 25-30 min. The absorbance at 450 nm was measured using a microplate reader. The ISM1 concentration in pg/ml was calculated from the standard curve.

### Quantification and Statistical Analysis

Data Representation and Statistical analysis. Replicates are described in the figure legends. All values in graphs are presented as mean+/−S.E.M. Values for n represent biological replicates for cell experiments or individual animals for in vivo experiments. For animal experiments, n corresponds to the number of animals per condition. Specific details for n values are noted in each figure legend. Each cellular experiment using primary cells was repeated using at least two cohorts of mice. Mice were randomly assigned to treatment groups for in vivo studies. Each animal experiment was repeated using at least two cohorts of mice. Student's t test was used for single comparisons. Significant differences between two groups (* p<0.05, ** p<0.01, *** p<0.001) were evaluated using a two-tailed, unpaired t-test as the sample groups displayed a normal distribution and comparable variance. Two-way ANOVA with repeated measures was used for the body weights, hyperinsulinemic-euglycemic clamps, GTT and ITT. Human expression data was analyzed using Mann-Whitney test (* p<0.05, ** p<0.01, *** p<0.001).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for the treatment of a glucose and lipid-associated disorder in an individual, the method comprising:
administering an effective dose of an Isthmin-1 (ISM1) agent to the individual, in a dose effective to reduce symptoms of the glucose and lipid-associated disorders.

2. The method of claim 1, wherein the glucose and lipid-associated disorder is one or both of type 2 diabetes and fatty liver disease.

3. The method of claim 2, wherein the fatty liver disease is non-alcoholic fatty liver disease (NAFLD) or nonalcoholic steatohepatitis (NASH).

4. The method of claim 1, wherein hyperglycemia and hyperlipidemia are simultaneously treated.

5. The method of claim 1, wherein the methods provides for a reduction of at least 5% in one or more disease indicia including reduction of liver weight or mass; reduction of blood glucose; reduced hepatic Fatty Acid Synthase (FAS) protein levels; and reduction of hepatic steatosis.

6. The method of claim 1, wherein administration is parenteral.

7. The method of claim 1, wherein the ISM1 agent is ISM1 protein, or a variant thereof.

8. The method of claim 1, wherein the individual is human.

9. The method of claim 1, wherein the ISM1 agent is mature human ISM1.

10. The method of claim 1, wherein the effective dose of an ISM1 agent is at least 0.1 mg/kg.

11. The method of claim 1, wherein administration is repeated daily, every 2 days, every 3 days, twice weekly, or weekly.

12. The method of claim 1, wherein the ISM1 agent is administered in combination with a second agent useful in treating a glucose and lipid-associated disorder.

13. The method of claim 12, wherein the second agent useful in treating a glucose and lipid-associated disorder is selected from metformin; a sulfonylurea; glinides; thiazolidinediones; DPP-4 inhibitor; GLP-1 receptor agonists; and SGLT2 inhibitor.

14. A pharmaceutical composition for use in the method of claim 1.

15. A pharmaceutical formulation comprising an effective dose of mature human ISM1 protein, in a unit dose effective for treatment of a glucose and lipid-associated disorder.


SEQUENCE LISTING (1 sequence)

SEQ ID NO: 1 - protein, 464 aa, Homo sapiens
MVRLAAELLLLLGLLLLTLHITVLRGSGAADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDEDYIKQFQEAREY